(19) United States
(12) Patent Application Publication
(10) Pub. No.: US 2026/0250721 A1
(43) Pub. Date: Aug. 27, 2026

(54) Biological Anisole Production

(71) Applicant: Wageningen Universiteit, Wageningen (NL)

(72) Inventors: Lyon Bruinsma, Wageningen (NL); Christos Batianis, Utrecht (NL); Vitor Alexandre Pires Martins Dos Santos, Wolfenbüttel (DE)

(21) Appl. No.: 19/489,687

(22) PCT Filed: Jun. 5, 2024
(86) PCT No.: PCT/NL2024/050290

§ 371 Date: Dec. 3, 2025

(30) Foreign Application Priority Data
Jun. 5, 2023 (EP) 23177385.4
Sep. 28, 2023 (EP) 23386089.9

(51) Int. Cl. C12P 7/22; C12N 1/16; C12N 1/20; C12N 9/10; C12N 9/88; C12N 15/52; C12R 1/01; C12R 1/40; C12R 1/645

(52) U.S. Cl. CPC C12P 7/22; C12N 1/16; C12N 1/20; C12N 9/1007; C12N 9/88; C12N 15/52; C12R 2001/01; C12R 2001/40; C12R 2001/645; C12Y 201/01025; C12Y 201/01146; C12Y 401/01061

(57) ABSTRACT

The invention is directed to methods for the production of anisole by a microorganism. The invention further is directed to a phenol O-methyltransferase enzyme and to the use of a phenol O-methyltransferase enzyme for the production of anisole by a microorganism.

### FIELD

The invention relates to industrial biotechnology. More specifically, the invention relates to the metabolic engineering of a microorganism for the microbial production of anisole.

### INTRODUCTION

The vast majority of industrially valuable chemicals are produced through chemical synthesis. Although efficient, many compounds needed for this synthesis are still petroleum derived. This approach is highly unsustainable and comes with many environmental issues (Mori, 2023. RSC Sustainability 1: 179-212). Therefore, sustainable alternative synthesis methods are desired. In recent years, microbial cell factories have gained considerable interest in the biobased production of chemicals (Nielsen and Keasling, 2016. Cell 164: 1185-1197). Microorganisms can use renewable start materials and are more selective, efficient and less environmentally hazardous compared to traditional chemical synthesis (Rosenberg et al., 2019. Trends Biotechnol 37: 29-37). For this reason, microbial cell factories, such as Escherichia coli, Saccharomyces cerevisiae and Pseudomonas putida are constantly being metabolically expanded to produce chemicals non-native to the host (Batianis et al., 2022. Metabol Eng 75: 47-57; Luo et al., 2019. PNAS USA 166: 10749-10756); Zhang et al., 2022. Nature 609: 341-347). Although promising, the use of microbial cell factories is often limited to the known biochemical space, whereas many industrially relevant compounds can still be produced only through chemical synthesis. However, many relevant compounds are only a single reaction step away from a known biological or bioactive compound (MohammadiPeyhani et al., 2022. Nature Commun 13: 560).

Recently, underground metabolism has been highlighted as a way to establish novel metabolic pathways (Rosenberg et al., 2019. Trends Biotechnol 37: 29-37). It relies on the promiscuous activity of enzymes, which can be explored to establish novel biochemistries. Although many chemical processes could be turned biological, often no enzymes are known for their conversion. To bridge this knowledge gap, retrosynthesis has been established as a means to identify novel biosynthetic pathways (Lin et al., 2019. Curr Opin Syst Biol 14: 82-107). These methods rely on enzymatic reaction rules, which describe the pattern of the reactive sites of compounds that are recognized by promiscuous enzymes (Hadadi et al., 2019. PNAS USA 116: 7298-7307). In this way, retrosynthesis can predict novel biosynthetic pathways for uncommon or new-to-nature compounds, moving them from the chemical to the biological space.

Anisole is an aromatic ether found in certain plants where it contributes to floral scents (Maia et al., 2014. Phytochemistry 103: 67-75), and it is also a key industrial intermediate product in the manufacturing of perfumes, dyes, drugs, fabric softeners, pesticides, water treatment and photographic chemicals (Dang et al., 2016. Chinese J Catal 37: 720-726). Anisole is usually synthesized by liquid phase processes in an alkaline environment (Vani et al., 2021. J Solut Chem 50: 160-177). However, these processes have become restricted in recent years, as the alkali are harmful to the environment. Moreover, the raw chemicals used in these processes are extremely toxic and dangerous to human health [(Dang et al., 2016. Chinese J Catal 37: 720-726). Currently, a greener method is used in which anisole is synthesized by vapour phase methylation. However, apart from anisole, this can lead to side products (impurities) such as o-cresol, p-cresol and p-xylene (Vani et al., 2021. J Solut Chem 50: 160-177). Moreover, in this process, phenol is the used substrate and is still predominantly produced from petroleum-derived feedstocks (Weber, 2020. Phenol. In: Ullmann's Encyclopedia of Industrial Chemistry, Electronic Release, Wiley-VCH, Weinheim). Therefore, a more efficient and cleaner process is desirable.

### BRIEF DESCRIPTION OF THE INVENTION

For the first time, biological anisole production in a microbial cell factory was established. A modular engineering approach was followed to establish anisole production using glucose as the sole feedstock. In addition, a growth-coupled selection system was constructed to optimize the kinetic parameters of CVOMT1 towards phenol as a substrate. Using this system, a new significantly more efficient variant was created with a higher affinity towards phenol, laying the foundation to create a kinetically fast phenol O-methyltransferase enzyme for the bioproduction of anisole. Overall, this work demonstrates the power of microbial cell factories to replace fully chemical processes with bio-logical ones. According to Axelrod & Daly (Axelrod and Daly, 1968. Biochim Biophys Acta 25: 472-478), anisole can be biologically produced from phenol by the enzyme phenol O-methyltransferase (EC 2.2.25). Although this enzyme is known to be present in mammals, currently no coding sequence is known. In a previous study by Pham (Thesis 2021; Evaluating and deploying genome-scale metabolic models for microbial cell factories. Wageningen University), the Selenzyme tool (Carbonell et al., 2018. Bioinform 34: 2153-2154) was deployed to search for promiscuous enzymes that could act as a phenol O-methyltransferase. Using this approach, they identified the CVOMT1 enzyme from Ocimum basilicum (Gang et al., 2002. Plant Cell 14: 505-519), which may be able to also act as phenol O-methyltransferase. CVOMT1 is a chavicol O-methyltransferase (EC 2.1.1.146) which transfers the methyl group from S-adenosyl-L-methionine (SAM) to the hydroxyl group on the benzene ring of chavicol, producing estragole.

The invention provides a method for the production of anisole by a microorganism, comprising providing said microorganism with a phenol O-methyltransferase enzyme and with a source of S-adenosyl-L-methionine; incubating the microorganism in a growth medium in the presence of a phenol source; to thereby have anisole produced by the microorganism. Said source of S-adenosyl-L-methionine preferably is methionine. Said phenol source preferably is 4-hydroxybenzoate (4HB).

In methods of the invention, the microorganism may be further provided with a 4HB decarboxylase, such as a 4HB decarboxylase from Bacillus subtilis.

In methods of the invention, the phenol source may be a sugar such as glucose and the microorganism may be further provided with a chorismate pyruvate lyase enzyme. Said chorismate pyruvate lyase preferably is a feed-back-resistant gene such as ubiCE31Q/M34V from E. coli.

In methods of the invention, the microorganism may lack functional expression of a 3-dehydroshikimate (DHS) dehydratase, a pyruvate kinase, preferably all functional pyruvate kinases, a hydroxybenzoate hydroxylase, a phosphoenolpyruvate carboxylase, or a combination thereof.

In methods of the invention, the phenol O-methyltransferase enzyme may have at least 70% sequence identity to a chavicol O-methyltransferase (EC 2.1.1.146).

In methods of the invention, the phenol O-methyltransferase enzyme may have at least 70% sequence identity to SEQ ID NO:1, preferably comprising a D264N amino acid alteration.

In methods of the invention, an organic solvent may be added to the growth medium to capture the produced anisole. Said organic solvent preferably comprises or is dodecane, ethyl acetate, or a mixture thereof.

In methods of the invention, the microorganism may be a bacterium such as Escherichia coli or Pseudomonas putida, or a yeast such as Saccharomyces cerevisiae.

The invention further provides a phenol O-methyltransferase enzyme (POMT1) comprising the amino acid sequence of SEQ ID NO:1 comprising a D264N amino acid alteration.

The invention additionally provides a nucleic acid molecule that encodes a POMT1, preferably a POMT1 comprising the amino acid sequence of SEQ ID NO:1 comprising a D264N amino acid alteration.

The invention further provides a microorganism comprising the nucleic acid molecule that encodes POMT1, preferably a POMT comprising the amino acid sequence of SEQ ID NO:1 comprising a D264N amino acid alteration.

The invention further provides a use of a phenol O-methyltransferase enzyme for the production of anisole by a microorganism, whereby said microorganism is incubated in the presence of a source of S-adenosyl-L-methionine, preferably in the presence of methionine. Said phenol O-methyltransferase enzyme preferably has at least 70% sequence identity to SEQ ID NO:1, preferably comprising a D264N amino acid alteration. Said use may involve the growth of the microorganism in the presence of a sugar, such as glucose and the provision of the microorganism with a chorismate pyruvate lyase. Said microorganism optionally lacks functional expression of a 3-dehydroshikimate (DHS) dehydratase and a pyruvate kinase, preferably of all functional pyruvate kinases.

### FIGURE LEGENDS

FIG. 1. Enzymatic conversion by CVOMT1 from Ocimum basilicum. A) Native chavicol O-methyltransferase activity of CVOMT1 converting chavicol to estragole. B) Low promiscuous activity of CVOMT1 converting phenol to anisole. Relative activities are derived from Gang et al., 2002. Plant Cell 14: 505-519.

FIG. 2. Toxicity analyses. A) Growth curves of P. putida KT2440 subjected to different phenol concentrations. B) Growth curves of P. putida KT2440 subjected to different anisole concentrations. Growth curves represent the mean value±SD from three independent experiments.

FIG. 3. Anisole production by CVOMT1. A) Schematic representation of phenol-O-methyltransferase activity by CVOMT1. B) Phenol consumption in 24 hours. C) Anisole production after 24 hours. Abbreviations: (SAM), S-adenosyl-l-methionine, (SAH), S-adenosyl-L-homocysteine, (MET), methionine, (EV), empty vector. Data points and bar graphs represent the mean value±SD from three independent experiments. ****, p<0.0001 determined by an unpaired Student's t-test.

FIG. 4. Anisole production from 4-hydroxybenzoate (4HB). A) Schematical representation of anisole production from 4HB. Phenol is produced by the 4HB decarboxylase from Bacillus subtilis. Anisole is produced by the chavicol O-methyltransferase from Ocimum basilicum. B) Phenol production from HB over the course of two days. C) Anisole production from 4HB over the course of four days. Abbreviations: (4HB), 4-hydroxybenzoate, C02, (carbon dioxide), (SAM), S-adenosyl-l-methionine, (SAH), S-adenosyl-L-homocysteine, (MET), methionine. Bar graphs represent the mean value±SD from three independent experiments.

FIG. 5. 4-Hydroxybenzoate (4HB) production from glucose. A) Schematic representation of the 4HB production pathway in P. putida KT2440. Mutations are denoted by the red square-dotted arrows. Overexpressed genes are denoted by green arrows. B) Aromatic yields detected during 4HB production from glucose. Abbreviations: (GLUC), glucose, (E4P), erythrose-4-phosphate, (PEP) phosphoenolpyruvate, (CHA), chorismate, (PYR), pyruvate, (4HB), 4-hydroxybenzoate, (PCA), protocatechuate, (AcCoA), acetyl-CoA, (OAA), oxaloacetate. Bar graphs represent the mean value±SD from three independent experiments.

FIG. 6. Anisole production from glucose. A) Schematic representation of the anisole production pathway in P. putida KT2440. B) Aromatic titers detected during anisole production from glucose. Abbreviations: (GLUC), Glucose, (CHA), Chorismate, (4HB), 4-hydroxybenzoate, (PYR), pyruvate, (SAM), S-adenosyl-l-methionine, (SAH), S-adenosyl-L-homocysteine, (MET), methionine. Bar graphs represent the mean value±SD from three independent experiments.

FIG. 7. Construction of a cysteine and methionine auxotroph. A) Schematic representation of the constructed knockouts. B) Growth curves of CM-Aux. Growth can only occur when both the amino acids cysteine and methionine are added to the media. Abbreviations: (GLUC), glucose, (CYS), cysteine, (MET), methionine, (hCYS), homocysteine, (CYST), cystathionine, (HSER), homoserine, (SER), serine. Growth curves represent the mean value±SD from three independent experiments.

FIG. 8. Establishing a growth-coupled methyltransferase selection system A) Selection of the C-Module in CM-Aux. B) Growth curves of CM-Aux with the implemented C-Module. C) Selection of the C and A-Module in CM-Aux. D) Growth curves of CM-Aux with the implemented C and A-Module. Abbreviations: (GLUC), glucose, (CYS), cysteine, (MET), methionine, (hCYS), homocysteine, (CYST), cystathionine, (ASP), aspartate, (SER), serine, (Phen), phenol, (Anis), anisole, (N/A), not applicable, (DT), doubling time, (h) hours. Growth curves represent the mean value±SD from three independent experiments.

FIG. 9. Optimization of the parameters for growth coupled selection. A) Heatmap of the specific growth rates of CM-Aux expressing the C and A-Module at different methionine and phenol concentrations. B) Heatmap of the final acquired cell densities of CM-Aux expressing the C and A-Module at different methionine and phenol concentrations. All values represent the average from three independent experiments.

FIG. 10. A) Anisole distribution in the two phases after 1 h incubation. B) Phenol distribution in the two phases after 1 h incubation. Bar graphs represent the mean value±SD from three replicates. C) Anisole distribution at different concentration of dodecane tested. Bar graphs represent the mean value±SD from three replicates. Bar graphs represent the mean value±SD from three replicates.

FIG. 11. A) Specific growth rates of CM-Aux equipped with CVOMT or POMT at different phenol concentrations B) Phenol consumption and anisole production of Empty vector vs CVOMT1 vs POMT1. Bar graphs represent the mean value±SD from three different replicates

### DETAILED DESCRIPTION OF THE INVENTION

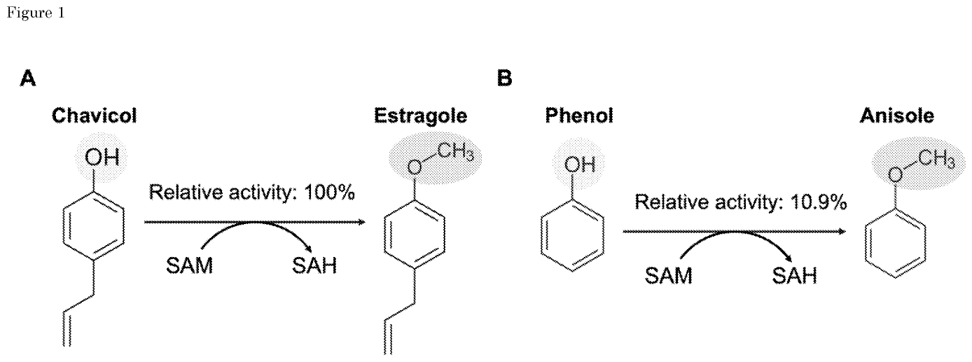

The invention is a novel biological method for producing anisole, an aromatic ether, utilizing a genetically engineered microbial cell factory. Central to this invention is the use of the enzyme CVOMT1 from Ocimum basilicum, which has been optimized through directed evolution to perform with high efficiency on the substrate phenol, producing anisole as a result (FIG. 1).

Anisole is a key intermediate in the perfume and fragrance industry for the manufacture of perfumes due to its aromatic ether characteristics; in the pharmaceutical industry for the synthesis of various pharmaceuticals; in dye manufacturing, where it plays a role in the manufacturing of certain dyes; in pesticide production, where it is used in the creation of certain pesticides; in water treatment processes; and in the production of certain photographic chemicals.

The current industrial methods of anisole production, such as liquid phase processes and vapor phase methylation, use environmentally harmful chemicals and petroleum-derived feedstocks. These methods raise serious environmental concerns due to their significant carbon footprints and potential for causing pollution.

A sustainable, microbial-based production method for anisole could significantly benefit these industries, especially with the growing consumer preference for environmentally friendly products. The adoption towards a biotechnological method for anisole production could reduce reliance on petroleum-derived feedstocks and harmful chemical processes, thereby contributing to sustainability goals and possibly providing a competitive edge in a market increasingly conscious of environmental impact.

The present method is based on two elements: (1) A genetically engineered microbial cell factory, which provides the host for enzymatic activities; and (2) The use of the enzyme CVOMT1 from Ocimum basilicum, which has been repurposed to act as phenol O-methyltransferase, converting phenol into anisole.

These elements differentiate this invention from the prior art as they provide a novel biological solution to the production of anisole, an area that has been traditionally dominated by chemical synthesis methods and has seen little progress in biological approaches due to the lack of efficient and well-characterized enzymes for the conversion of sustainable precursors to anisole.

The invention is directed towards a biological method for the production of anisole by a microorganism. Said microorganism may be a bacterium, e.g., an Escherichia species such as E. coli; a Pseudomonas species such as P. putida; a Clostridium species such as C. butyclicum; a Xanthomonas species such as X. campestris; a Corynebacterium species such as C. glutamicum; or a Deinococcus species such as D. radiorans. Alternatively, said microorganism may be a yeast, e.g., a Saccharomyces species such as S. cerevisiae; or an Aspergillus species such as A. niger; an alga such as Chlamydomonas reinhardtii, Dunaliella salina, Hematococcus pluvialis, Nitzschia closterium, Porphyridium cruentum, Crypthecodinium cohnii, Phaeodactylum tricornutum, or Nannochloropsis gaditana; or a cell line such as a mammalian cell line. Other organisms with a history of safe use in industrial biotechnology can also be explored.

Growth media for the growth of a microorganism may include aqueous media comprising at least one carbon source such as a sugar, preferably a monosaccharide such as fructose and glucose, a disaccharide such as lactose and maltose, or a mixture thereof, and/or glycerol. Said at least one carbon source may be present between 0.1% and 30% (w/w), more preferably between 0.5% and 2% (w/w).

Said growth medium may further comprise one or more of magnesium (e.g., MgCl₂ and/or MgSO₄), manganese (e.g., MnCl₂), calcium (e.g., CaCl₂), sulfur, and ions of the trace elements selenium, vanadium and zinc. These inorganic salts and trace elements may be obtained commercially, for example from Sigma (Saint Louis, Missouri).

Said growth medium may further comprise a chemical buffering system such as Na₂HPO₄ and KH₂PO4, 2-[4-(2-hydroxyethyl)piperazin-1-yl]ethanesulfonic acid (HEPES), or a carbonate-based buffer, in order to keep the pH between 6.8 and 7.4. A preferred buffering system is provided by Na₂HPO₄ and KH₂PO₄. A pH indicator, such as phenol red, may be added that turns yellow in acidic conditions (<6.8), and pink in basic conditions (>8.2). Incubation pH may be controlled by the high CO2 levels in the incubator of 5% or more.

Said growth medium may further include one or more vitamins such as thiamin, nicotinic acid, pyridoxal, riboflavin, folic acid, pantetheine, or a combination thereof such as nicotinic acid and thiamin, nicotinic acid and pyridoxal, nicotinic acid and riboflavin, nicotinic acid and folic acid, or nicotinic acid and pantetheine. These vitamins may be commercially obtained, for example from Sigma (Saint Louis, Missouri).

Said growth medium may further comprise nucleotides, including, for example, nucleoside triphosphates such as adenosine triphosphate, guanosine triphosphate, cytidine triphosphate, uridine triphosphate, or a combination thereof. Said nucleotides preferably include adenosine ribonucleotide triphosphate and adenosine deoxyribonucleotide triphosphate, collectively termed herein adenine; guanosine ribonucleotide triphosphate and guanosine deoxyribonucleotide triphosphate, collectively termed herein guanine; cytidine ribonucleotide triphosphate, termed cytidine; cytidine deoxyribonucleotide triphosphate, termed deoxycytidine; thymidine deoxyribonucleotide triphosphate, termed thymine; uridine ribonucleotide triphosphate, termed uracil; adenosine ribonucleotide 3′,5′-bisphosphate, or a combination thereof. These nucleotides may be obtained commercially, for example from Sigma (Saint Louis, Missouri).

Said nucleotides, may be present in a growth medium at a concentration between 1 micromolar (μM) and 1 millimolar (mM), more preferably at a concentration between 50 μM and 200 μM.

Said growth medium may further comprise amino acids, such as the essential amino acids histidine, isoleucine, leucine, lysine, methionine, phenylalanine, threonine, tryptophan, valine, or a combination thereof. Said growth medium may comprise all 21 natural (L) amino acids alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine and valine, or combinations thereof, preferably alanine, arginine, asparagine, cysteine, glutamine, glycine, histidine, isoleucine, leucine, lysine, methionine, phenylalanine, proline, serine, threonine, tryptophan, tyrosine and valine, without aspartic acid, selenocysteine and glutamic acid. These amino acids may be obtained commercially, for example from Sigma (Saint Louis, Missouri).

Said amino acids preferably are present in a growth medium at concentrations between 0.001 g/L and 0.1 g/L.

Said growth medium may further contain one or more antibiotics, such as penicillin, streptomycin, or a mixture thereof.

A preferred growth medium for a bacterium such as Pseudomonas putida is M9 minimal medium, which is commercially available.

Said growth medium may comprise an organic solvent to capture the produced anisole in a two phase (diphasic) culture system, for example as described in Wierckx et al., 2005 (Wierckx et al., 2005. Appl Environ Microbiol 71: 8221-8227). Said organic solvent may be any organic solvent that is compatible with growth of the microorganism and production of anisole. A preferred organic solvent comprises, or is, dodecane, ethyl acetate, octanol, tributyrin, oleyl alcohol, or a mixture thereof.

Said microorganism functionally expresses a phenol O-methyltransferase enzyme (EC 2.2.25; S-adenosyl-L-methionine:phenol O-methyltransferase). This enzyme is known to be localized in the microsomes of the liver and lung of mammals and is also present in other tissues (Axelrod and Daly, 1968. Biochim Biophys Acta 25: 472-478). However, no sequences have been reported up to date. Previous studies (Pham (Thesis 2021; Evaluating and deploying genome-scale metabolic models for microbial cell factories. Wageningen University), have suggested that the CVOMT1 enzyme from Ocimum basilicum (Gang et al., 2002. Plant Cell 14: 505-519) may be able to also act as phenol O-methyltransferase. The inventors have now shown that this enzyme, in the presence of S-adenosyl-L-methionine (SAM), may act as a phenol O-methyltransferase.

A phenol O-methyltransferase enzyme thus may have at least 70% sequence identity to a chavicol O-methyltransferase (EC 2.1.1.146), such as the CVOMT1 enzyme from Ocimum basilicum, provided herein as SEQ ID NO:1. Said percentage of identity preferably is at least 80% to SEQ ID NO:1, at least 90%, at least 92%, at least 95%, or at least 99%.

Said phenol O-methyltransferase enzyme may have a D264N amino acid alteration, when compared to the sequence of SEQ ID NO:1. As is shown herein below, said D264N amino acid alteration may induce a conformational change, allowing increased binding affinity of the enzyme towards phenol, when compared to an un-altered enzyme.

The growth medium of the microorganism for the production of anisole comprises a source of S-adenosyl-L-methionine (SAM). Said SAM is required as a cofactor for the efficient conversion of phenol to anisole. SAM may be produced from ATP and methionine by an S-adenosylmethionine synthetase enzyme. SAM may be produced by the microorganism from a carbon source through methionine via a transsulfuration pathway, or a direct-sulfurylation pathway, as is known to a person skilled in the art. As an alternative, methionine, the direct precursor to SAM biosynthesis, may be added to the growth medium as a source of S-adenosyl-L-methionine. Methionine may be added at a concentration between 0.01 mM and 10 mM, preferably about 1 mM.

Phenol is predominantly produced by three different biosynthetic pathways which are derived from the shikimate pathway. A first reaction is performed by tyrosine phenol-lyase, in which tyrosine is cleaved to pyruvate, phenol, and ammonia. A second reaction involves the conversion of chorismate into isochorismate, for example by an isochorismate synthase, then the conversion of isochorismate into salicylate, for example by an salicylate synthase, or an isochorismate pyruvate-lyase, such as pyochelin biosynthetic protein (pchB) from Pseudomonas aeruginosa, and finally the conversion of salicylate into phenol by a salicylate decarboxylase. A third reaction produces phenol through the decarboxylation of 4HB by 4HB decarboxylase. As tyrosine production contains more enzymatic steps and its biosynthesis is tightly regulated, a convenient phenol source that is present in the growth medium therefore is 4HB. 4HB may be present in the growth medium at a concentration between 0.01 mM and 10 mM, preferably about 1 mM.

The decarboxylation of 4HB may involve expression of a 4HB decarboxylase. If not expressed in the microorganism, it may be exogenously expressed in the microorganism. A suitable 4HB decarboxylase is a 4HB decarboxylase from Bacillus subtilis, which is herein provided as SEQ ID NO:5, or a sequence having at least 70% sequence identity to SEQ ID NO:5 over the whole length. Said exogenous expression may be provided by transforming the microorganism with an expression construct that expresses said 4HB decarboxylase. Said expression construct further provides elements, such as promoter regions, that direct expression of the 4HB decarboxylase. Expression constructs for expression of an enzyme in a microorganism as claimed are known to the skilled person.

As an alternative, a construct that expresses 4HB decarboxylase may be integrated into the genome of the microorganism by a site-specific recombinase, as is known to a person skilled in the art. Said site-specific recombinase preferably is selected from a Zinc finger nuclease, a transcription activator-like effector nuclease (TALEN), a topoisomerase I-like recombinase such as Cre recombinase from the P1 bacteriophage, a Saccharomyces cerevisiae-derived flippase (Flp recombinase), a lambda integrase, a gamma-delta resolvase, Tn3 resolvase, φC31 integrase and/or a clustered regularly interspaced short palindromic repeats (CRISPR)-guided nuclease. Preferred site-specific recombinases are a Zinc finger nuclease, a transcription activator-like effector nuclease (TALEN), a clustered regularly interspaced short palindromic repeats (CRISPR)-guided nuclease, or a combination thereof.

In methods of the invention, a p-hydroxybenzoate hydroxylase, which is responsible for 4HB degradation to protocatechuate (PCA), may be functionally inactivated in the microorganism. Methods to functionally inactivate an enzyme include the genomic alteration of the gene encoding the enzyme, including deletion of part or the complete gene. Deletion of part or the complete gene encoding a p-hydroxybenzoate hydroxylase may be accomplished by homologous recombination, or by a site-specific recombinase as is indicated herein above. An example of a hydroxybenzoate hydroxylase is provided by the product of the pobA gene of P. putida, which is herein provided as SEQ ID NO:7, or an enzyme having at least 70% sequence identity to SEQ ID NO:7 over the whole length.

A sugar such as glucose, an alcohol such as glycerol, or an organic acid such as acetic acid, may be provided as an alternative phenol source for 4HB to a microorganism. In case a sugar such as glucose, an alcohol such as glycerol, or an organic acid such as acetic acid, is included in the growth medium as phenol source, the microorganism preferably expresses a chorismate pyruvate lyase. This enzyme cleaves chorismate, the final product of the shikimate pathway, equimolarly to pyruvate and 4HB. A suitable chorismate pyruvate lyase is encoded by the gene ubiC in E. coli. An example of such enzyme is herein provided as SEQ ID NO:4 (UniProt P26602), or an enzyme having at least 70% sequence identity to SEQ ID NO:4 over the whole length. Feedback resistant mutants of UbiC have been described (Jha et al., 2019. ACS Synth Biol 8: 775-786), and are suited for expression in a microorganism for the production of anisole from a sugar as phenol source. Said feedback-resistant chorismate pyruvate lyase preferably is ubiCE31Q/M34V from E. coli (Jha et al., 2019. ACS Synth Biol 8: 775-786), wherein the amino acid alteration E31Q and M34V correspond to E32Q and M35V of SEQ ID NO:4.

Said microorganism may further express a phospho-2-dehydro-3-deoxyheptonate aldolase, such as a phospho-2-dehydro-3-deoxyheptonate aldolase that is not feedback-inhibited by phenylalanine. Said enzyme catalyzes the conversion of D-erythrose 4-phosphate+H2O+phosphoenolpyruvate into 7-phospho-2-dehydro-3-deoxy-D-arabino-heptonate+phosphate, and may be used to push the flux in the shikimate pathway. A suitable phospho-2-dehydro-3-deoxyheptonate aldolase is provided by the aroG gene product of E. coli, which is provided herein as SEQ ID NO:6, or an enzyme having at least 70% sequence identity to SEQ ID NO:6 over the whole length. A phenylalanine-resistant mutant is provided by a D146N amino acid alteration of SEQ ID NO:6.

In case a sugar such as glucose, an alcohol such as glycerol, or an organic acid such as acetic acid, is included in the growth medium as phenol source, the microorganism may, in addition to the expression of a chorismate pyruvate lyase, lack functional expression of a 3-dehydroshikimate (DHS) dehydratase and/or of a pyruvate kinase, preferably of all functional pyruvate kinases. DHS dehydratase converts the shikimate pathway intermediate 3DHS into PCA, diverting the flux away from 4HB production. Hence, functionally inactivation of this enzyme in a microorganism will increase production of anisole by the microorganism, when the phenol source is a sugar, such as glucose. Methods to functionally inactivate an enzyme include the genomic alteration of the gene encoding the enzyme, including deletion of part or the complete gene. Deletion of part or the complete gene encoding a DHS dehydratase may be accomplished by a site-specific recombinase as is indicated herein above. A DHS dehydratase gene in Pseudomonas putida is quiC, which preferably is functionally inactivated in P. putida to increase production of anisole by P. putida when the phenol source is a sugar such as glucose.

Said microorganism may further lack functional expression of a pyruvate kinase. This enzyme cleaves phosphoenolpyruvate (PEP) to pyruvate, of which two moles and one mole of erythrose-4-phosphate (E4P) are required in the shikimate pathway to synthesize chorismate, the final product and precursor to 4HB. To increase PEP availability, one or more pyruvate kinases may be functionally inactivated. Hence, functionally inactivation of this enzyme in a microorganism will increase production of anisole by the microorganism, when the phenol source is a sugar such as glucose, an alcohol, such as glycerol, or an organic acid, such as acetic acid. Methods to functionally inactivate an enzyme include the genomic alteration of the gene encoding the enzyme, including deletion of part or the complete gene. Deletion of part or the complete gene encoding a pyruvate kinase may be accomplished by a site-specific recombinase as is indicated herein above. Said pyruvate kinase genes in Pseudomonas putida include pyk and pykA.

As an alternative, or in addition, said microorganism may further lack functional expression of a phosphoenolpyruvate carboxylase, which catalyzes the addition of bicarbonate to phosphoenolpyruvate to form oxaloacetate and inorganic phosphate. Hence, functionally inactivation of this enzyme in a microorganism will increase production of anisole by the microorganism, when the phenol source is a sugar such as glucose, an alcohol such as glycerol, or an organic acid such as acetic acid.

The invention further provides a phenol O-methyltransferase enzyme (POMT1) comprising the amino acid sequence of SEQ ID NO:1 comprising a D264N amino acid alteration, or an amino acid sequence that has at least 70% sequence identity to SEQ ID NO:1 and comprises a D264N amino acid alteration, or a D-to-N alteration at a position corresponding to position 264 of SEQ ID NO:1. Methods to analyze whether an enzyme has phenol O-methyltransferase activity are known in the art, and include growing a microorganism in the presence of phenol and analyzing the production of anisole, as described herein.

The invention additionally provides a nucleic acid molecule that encodes a POMT1 as described herein above, preferably a POMT1 comprising the amino acid sequence of SEQ ID NO:1 comprising a D264N amino acid alteration. Said nucleic acid molecule may be a DNA molecule, an RNA molecule, or a mixture thereof.

The invention further provides a microorganism comprising a nucleic acid molecule that encodes a POMT1. Said microorganism preferably expresses said POMT1. Said nucleic acid molecule may be codon-optimized for expression in said microorganism. Said microorganism may be a bacterium, e.g., an Escherichia species such as E. coli; a Pseudomonas species, such as P. putida; a Clostridium species, such as C. butyclicum; a Xanthomonas species, such as X. campestris; a Corynebacterium species, such as C. glutamicum; or a Deinococcus species, such as D. radiorans. Said suitable microorganism may also be a yeast, e.g., a Saccharomyces species, such as S. cerevisiae, or an Aspergillus species, such as A. niger; an alga; or a cell line such as a mammalian cell line. Other organisms with a history of safe use in industrial biotechnology can also be explored.

The invention further provides a use of a phenol O-methyltransferase enzyme for the production of anisole by a microorganism, in the presence of a source of S-adenosyl-L-methionine, preferably in the presence of methionine. Said suitable microorganism may be a bacterium, e.g., an Escherichia species such as E. coli; a Pseudomonas species, such as P. putida; a Clostridium species, such as C. butyclicum; a Xanthomonas species, such as X. campestris; a Corynebacterium species, such as C. glutamicum; or a Deinococcus species, such as D. radiorans. Said suitable microorganism may also be a yeast, e.g., a Saccharomyces species, such as S. cerevisiae, or an Aspergillus species, such as A. niger; an alga; or a cell line such as a mammalian cell line. Other organisms with a history of safe use in industrial biotechnology can also be explored.

Said phenol O-methyltransferase enzyme may have at least 70% sequence identity to SEQ ID NO:1, and preferably comprises a D264N amino acid alteration.

Said microorganism may be grown in the presence of a sugar, such as glucose whereby the microorganism is further provided with a chorismate pyruvate lyase, and wherein the microorganism optionally lacks functional expression of a 3-dehydroshikimate (DHS) dehydratase and of a pyruvate kinase, preferably of all functional pyruvate kinases. Said microorganism optionally expresses a phospho-2-dehydro-3-deoxyheptonate aldolase, such as a phospho-2-dehydro-3-deoxyheptonate aldolase that is provided by the aroG gene product of E. coli which is provided herein as SEQ ID NO:6. A phenylalanine-resistant mutant is provided by a D146N amino acid alteration of SEQ ID NO:6.

Where reference is made herein to a specific sequence, a person skilled in the art will be able to identify the corresponding gene from another microorganism, for example, using existing databases such as NCBI, UniProt, or GenBank. For example, a skilled person can align a sequence of a gene or protein from Pseudomonas putida with the genetic information of another species to identify the corresponding gene. For example, a skilled person can identify a pobA gene from another organism as having the highest homology to the pobA gene as described in the present invention as SEQ ID NO: 7. A gene or gene product (protein) from another organism having at least 70% sequence identity to a sequence provided herein over the whole length, may be identified as the corresponding gene or product from that other organism. Said percentage of identity preferably is at least 80% to a sequence as provided herein, at least 90%, at least 92%, at least 95%, or at least 99%.

Alternatively, or in addition, a skilled person can identify a gene from another organism as the corresponding gene if it is located in the same genetic context, for example in the same bacterial operon. Bacterial operons are co-regulated gene clusters wherein genes are grouped under a common control mechanism. A gene in an organism that is present in the same operon as, for example, in Pseudomonas putida, especially in the same position in an operon, can be identified as the corresponding gene from another organism.

### EXAMPLES

### Example 1

### Materials and Methods

### Plasmids, Primers, and Strains

All strains used in the present study are listed in Table 1. Plasmids used in this study are listed in Table 2.

### Bacterial Strains and Growth Conditions

P. putida and E. coli cultures were incubated at 30° C. and 37° C. respectively. For cloning purposes, both strains were propagated in Lysogeny Broth (LB) medium containing 10 g/L NaCl, 10 g/L tryptone, and 5 g/L yeast extract. For the preparation of solid media, 1.5% (w/v) agar was added. Antibiotics, when required, were used at the following concentrations: kanamycin (Km) 50 μg/mL, gentamycin (Gm) 10 μg/mL, chloramphenicol (Cm) 50 μg/mL and apramycin (Apra) 50 μg/mL. All growth experiments were performed using M9 minimal medium (per Liter; 3.88 g K2HPO4, 1.63 g NaH2PO4, 2.0 g (NH4)2SO4, pH 7.0. The M9 media was supplemented with a trace elements solution (10 mg/L ethylenediaminetetraacetic acid (EDTA), 0.1 g/L MgCl2 6 H2O, 2 mg/L ZnSO4·7H2O, 1 mg/L CaCl₂)·2H2O, 5 mg/L FeSO4·7 H2O, 0.2 mg/L Na₂MoO4·2H2O, 0.2 mg/L CuSO4·5H2O, 0.4 mg/L CoCl2·6H2O, 1 mg/L MnCl2·2H2O). In these experiments, strains were precultured in 10 mL LB with corresponding antibiotics. Then, the cultures were washed twice in M9 media without a carbon source. Finally, the cultures were diluted to an OD600 of 0.1 to start the experiment. In all plate reader experiments, a glucose concentration of 10 mM was used. Unless otherwise indicated, the supplementary carbon sources, cysteine, methionine, homocysteine, and phenol were added at a concentration of 1 mM. Plate reader experiments were carried out in 200 μL of M9 medium using an ELx808 plate reader (BioTek). Growth (OD600) was measured over time using continuous shaking and measurements were taken every three minutes. Flask experiments were performed in 250 mL Erlenmeyer flasks filled with 25 mL of M9 minimal medium containing 50 mM of glucose. Unless otherwise indicated, the supplementary carbon sources, 4HB, phenol and methionine were added at a concentration of 1 mM. The cultures were incubated in a rotary shaker at 200 rpm at 30° C.

### Plasmid Construction

Plasmids were constructed using the previously described SevaBrick Assembly (Damalas et al., 2020. Microb Biotechnol 13: 1793-1806). All DNA fragments were amplified using Q5® Hot Start High-Fidelity DNA Polymerase (New England Biolabs). The cvomt1 gene from Ocimum basilicum was codon-optimized with the JCat tool (Grote et al., 2005. Nucleic Acids Res 33: W526-W531), and synthesized through Genescript (SEQ ID NO:2). The ubiC gene was amplified from the genomic DNA from E. coli and the feedback-resistant mutations were introduced during PCR. The feedback-resistant aroG gene was obtained from an in-house plasmid. The BsdBCD genes were amplified from the genomic DNA of Bacillus subtilis. All genes were expressed using the Biobrick BBa_J23100 promoter and BBa_B0034 RBS. For genomic integration, the phenol operon was amplified by PCR from the assembled plasmid and cloned into a pGNW containing lox sites. The T7 RNA polymerase and Cre recombinases were amplified from in-house plasmids. All plasmids were transformed using heat shock in chemically competent E. coli DH5α λpir and selected on LB agar with corresponding antibiotics. Colonies were screened through colony PCR with Phire Hot Start II DNA Polymerase (Thermo Fisher Scientific). Isolated plasmids were verified using Sanger sequencing (MACROGEN, Inc.) and subsequently transformed into P. putida via electroporation.

### Strain Construction

Genetic deletions in this study were performed using the protocol previously described by Wirth et al., 2020 (Wirth et al., 2020. Microb Biotech 12: 233-249). Homology regions of 500 bp were amplified up and downstream of the target gene from the genome of P. putida KT. Both regions were cloned into the non-replicative pGNW vector and propagated in E. coli DH5α λpir. Correct plasmids were transformed into P. putida by electroporation and selected on LB+Km plates. Successful co-integrations were verified by PCR. Hereafter, co-integrated strains were transformed with the pQURE6-H and transformants were plated on LB+Gm containing 2 mM 3-methylbenzoic acid (3-mBz). This compound induces the XylS-dependent Pm promoter, regulating the I-Scel homing nuclease that cuts the integrated pGNW vector. Successful gene deletions were verified by PCR and Sanger sequencing (MACROGEN, Inc). Hereafter, the pQURE6-H was cured by removing the selective pressure and its loss was verified by sensitivity to gentamycin.

### Analytical Methods

Cell growth was determined by measuring the optical density at 600 nm (OD600) using an OD600 DiluPhotometer spectrophotometer (IMPLEN) or a Synergy plate reader (BioTek Instruments). Analysis of glucose in supernatants was performed using high-performance liquid chromatography (HPLC) (Thermo Fisher Scientific) equipped with an Aminex HPX-87H column. The mobile phase was 5 mM of H2SO4 at a flow rate of 0.6 mL/min, the column temperatures were held at 60° C. and the compounds were detected using a Shodex RI-101 detector (Shodex). The amounts of produced 4HB, phenol and anisole were determined using HPLC (Shimadzu) with a C18 column (4.6 mm×250 mm) and a UV/vis detector set at 472 nm. The mobile phase consisted of Milli-Q water (A), 100 mM formic acid (B) and acetonitrile (C) with a flow rate of 1 mL/min at 30° C. Chromatographic separation of analytes was attained using the following gradient program: t=0-5 min: A-55%, B-10% and C-35% from t=5-10 min ramp to A-10%, B-10% and C-80% and held until 15 min. Then from t=15-16 min, the gradient was returned to A-55%, B-10% and C-35% and maintained isocratic for a total run time of 18 min. For quantification, calibration curves were prepared using pure standards (purity) purchased from Sigma-Aldrich.

### High Throughput Screening of CVOMT1 Varieties

The CVOMT1 library was generated using the GeneMorph II Random Mutagenesis Kit (Agilent Technologies) following the manufacturer's protocol. In short, 50 ng of cvomt1 DNA was taken as the template for error-prone PCR and amplified with the primers CVOMT_ALE_FW and CVOMT_ALE_RV (see Table 3). The error-prone PCR was carried out for 30 thermal cycles and the PCR product was gel purified and cloned into a pSEVAb vector. Next, the library was transformed by electroporation into the CM-Aux strain containing the C-module and grown overnight in LB media with the corresponding antibiotics. The next day, cells were washed twice with M9 minimal media without a carbon source. Then, cells were reinoculated at an OD600 of 0.1 in 10 mL of M9 containing 10 mM glucose, 1 mM methionine and 0.1 mM phenol and grown overnight. This culture was subsequently plated on M9 agar containing the same carbon sources. After 2 days of growth, 92 visible large colonies were picked and inoculated in a 96-well plate filled with 200 μL of LB supplemented with the appropriate antibiotics. Two wells contained CM-Aux with the unevolved CVOMT1 enzyme as positive control and two were left blank as a negative control. The plate was incubated for 24 hours at 30° C. in an ELX808 plate reader. The next day, 5 μL was transferred to a new 96-well plate containing 200 μL M9 with 10 mM glucose, 1 mM methionine and 0.1 mM phenol and incubated for 24 hours at 30° C. Growth (OD600) was measured every three minutes over time using continuous shaking.

### Modelling Anisole Production

iJN1462, the latest developed genome-scale model (GEM) of P. putida, was used for anisole production and added the reactions for phenol-O-methyltransferase, 4HB decarboxylase and tyrosine phenol-lyase. Flux balance analysis was used to calculate the maximum theoretical by setting anisole production as the objective.

### Structural Protein Analysis

Structural models of CVOMT1 and POMT1 were generated using the open-source software ColabFold (Mirdita et al., 2022. Nature Methods 19: 679-682). Visualization and analysis of the protein 3D structures were performed in the Mol* 3D viewer of the RCSB protein data bank.

### Statistical Analysis

All reported experiments are derived from independent biological replicates. Figures represent the mean values of corresponding biological triplicates and the standard deviation. The level of significance of the difference when comparing results was evaluated by an unpaired Student's t-test.

### Results

Pseudomonas putida is a Robust Host to Produce Aromatics Compounds

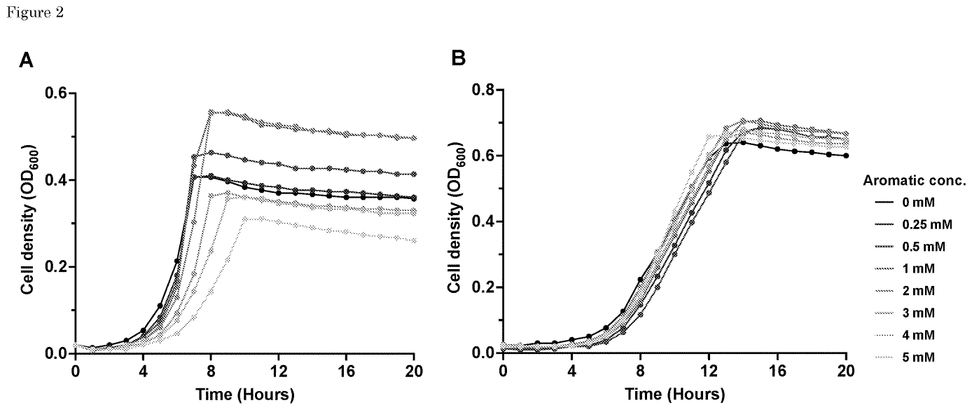

The production of aromatic compounds often comes with unwanted toxicity. One way of circumventing this is the utilization of microbial hosts with high tolerance towards aromatic compounds (Li et al., 2020. Biotechnol Adv 41: 107548). P. putida KT2440 is generally regarded as an excellent candidate, due to its inherent high tolerance to a range of toxic compounds including aromatics (Loeschcke and Thies, 2015. Appl Microbiol Biotechnol 99: 6197-6214). It is known to reconfigure its metabolic fluxes resulting in a significant surplus of NADPH as a response to combat oxidative stress (Nikel et al., 2021. SME Journal 15: 1751-1766). Phenol is a highly toxic chemical, and its presence generally poses a burden on the microbial vitality (Krastanov et al., 2013. Eng Life Sciences 13: 76-87; Wierckx et al., 2005. Appl Environ Microbiol 71: 8221-8227). Suppositiously, there is no data yet regarding anisole's microbial toxicity. Therefore, P. putida KT2440 was subjected to different phenol and anisole concentrations to examine the tolerance of the strain towards these two aromatics. The strain was able to withstand up to 5 mM of phenol (FIG. 2A).

Growth rates did not deviate up to 2 mM of phenol and even reached a higher cell density. However, at concentrations of 3 mM and above, phenol starts to have a detrimental effect, affecting growth rate and final cell density (FIG. 2A). On the contrary, the presence of anisole did not show any detrimental effects on growth (FIG. 2B). The presence of phenolic hydroxyl groups has been associated with the inhibition of microbial enzymes and the damage of the bacterial membrane (Miklasinska-Majdanik et al., 2018. Int J Environ Res Public Health 15: 2321). Since the hydroxyl group of phenol is replaced by a methyl group in anisole, the unwanted side effects might not exist.

### Testing CVOMT1 for Anisole Production

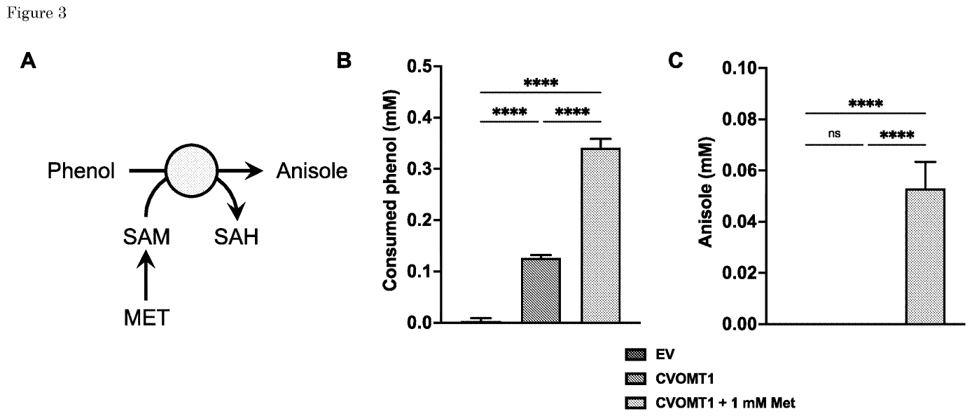

To examine the potential of CVOMT1 to establish anisole production, the cvomt1 gene was codon-optimized and placed it on a pSEVA23b backbone (Damalas et al., 2020. Microb Biotechnol 13: 1793-1806). P. putida KT2440 was transformed with this plasmid or an empty vector and the strains were fed with 50 mM glucose and 1 mM of phenol to determine anisole production. The empty vector control consumed 0.01 mM phenol after 24 hours, whereas CVOMT1 consumed 0.12 mM phenol (FIG. 3B). Although it was detected that CVOMT1 uses phenol as a substrate, no anisole was detected in the samples. For the efficient conversion of phenol to anisole, S-adenosyl-methionine (SAM) may be required as a cofactor. It was hypothesized that its availability might be limited, hampering the conversion of phenol. To increase SAM availability, 1 mM methionine was added to the media, the direct precursor to SAM biosynthesis (FIG. 3A). A drastically improved phenol consumption became detectible, and anisole production was observed (FIG. 3C). However, anisole yielded only 15.5% mol/mol of the converted phenol. Anisole is a volatile compound and most likely abiotically escapes the culture media. To examine this, three flasks containing 2 mM anisole in M9 minimal media were cultivated overnight. It was found that anisole indeed escaped the media, as 89% of the anisole disappeared overnight (data not shown).

Unfortunately, current strategies to capture anisole from fermentation broth are not yet known and require further research.

Establishing Anisole Production from Glucose

To achieve anisole production from glucose, an attempt was made to establish phenol production. Phenol is predominantly produced by two different reactions which both derive from the shikimate pathway. The first reaction is performed by tyrosine phenol-lyase, in which tyrosine is cleaved to form pyruvate, phenol, and ammonia. The second reaction produces phenol through the decarboxylation of 4-hydroxybenzoate (4HB). Flux balance analysis (FBA) was performed to determine which pathway would obtain higher yields. These analyses indicated that anisole production through tyrosine would obtain a slightly higher yield than through 4HB: 0.534 mol/mol compared to 0.531 mol/mol glucose, respectively. However, tyrosine production contains more enzymatic steps and its biosynthesis is tightly regulated (Miao et al. 2015. Appl Microbiol Biotech 99: 5163-5173). Moreover, the tyrosine phenol-lyase is feedback-inhibited by phenol, which would further limit anisole production (Li et al., 2020. Biotechnol Adv 41: 107548). Therefore, the 4HB variant was selected to establish phenol production. An attempt was made to clone the BscBCD operon from Bacillus subtilis, which encodes a 4HB decarboxylase complex (Lupa et al., 2008. Can J Microbiol 54: 75-81). However, as phenol production is quite toxic, plasmid-born expression of the operon was unsuccessful.

Therefore, a genomic landing pad based on the Cre/lox system was created. To allow high transcription levels similar to plasmid-borne expression, the T7-RNA polymerase and its depending promoter were integrated in the landing pad. The landing pad was integrated in KT2440 ApobA to assess phenol production. The pobA gene encodes a p-hydroxybenzoate hydroxylase which is responsible for 4HB degradation to protocatechuate (PCA). Therefore, in this setup, 4HB cannot be degraded and can only be converted towards phenol. The strain was supplemented with 50 mM glucose and 1 mM 4HB and phenol production was measured over time.

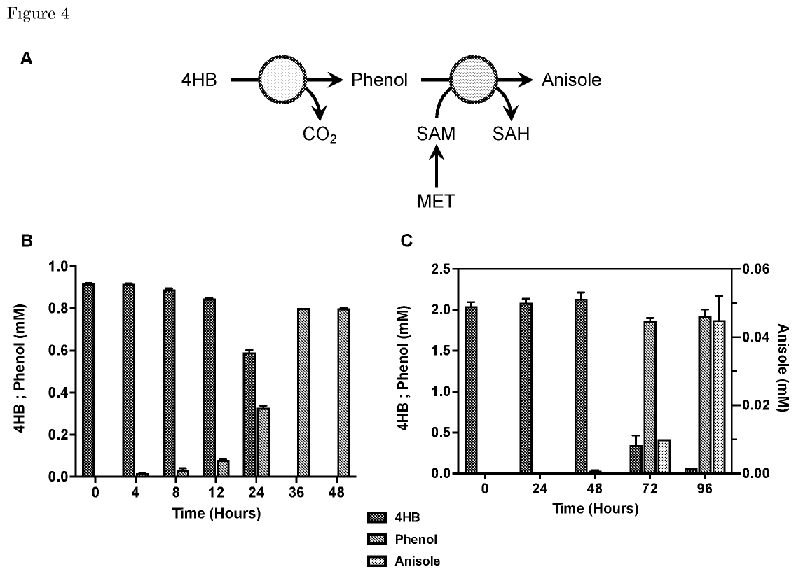

Although a hard restraint on growth and 4HB conversion was observed, most likely attributed to the toxicity of phenol, all 4HB was converted to phenol over the course of 2 days (FIG. 4B). With phenol production established, an aim was set to produce anisole using 4HB as the precursor. The cvomt1 gene and the 4HB decarboxylase were cloned in the same plasmid and transformed into KT2440 ApobA. Although the construction of the previous phenol module on a plasmid was unsuccessful due to phenol toxicity this was not observed for the anisole module. It is most likely that CVOMT1 is detoxifying the intracellular phenol towards the less toxic anisole, allowing plasmid-born expression. KT2440 ApobA was grown in 50 mM glucose supplemented with 2 mM of 4HB and methionine. The strains started to consume 4HB after 48 hours and within 24 hours the majority was converted towards phenol with minor production of anisole (0.01 mM). After 96 hours, almost all 4HB was converted towards phenol. However, a fraction of anisole could be detected (0.045 mM), demonstrating the successful production of anisole from 4HB as the precursor (FIG. 4C).

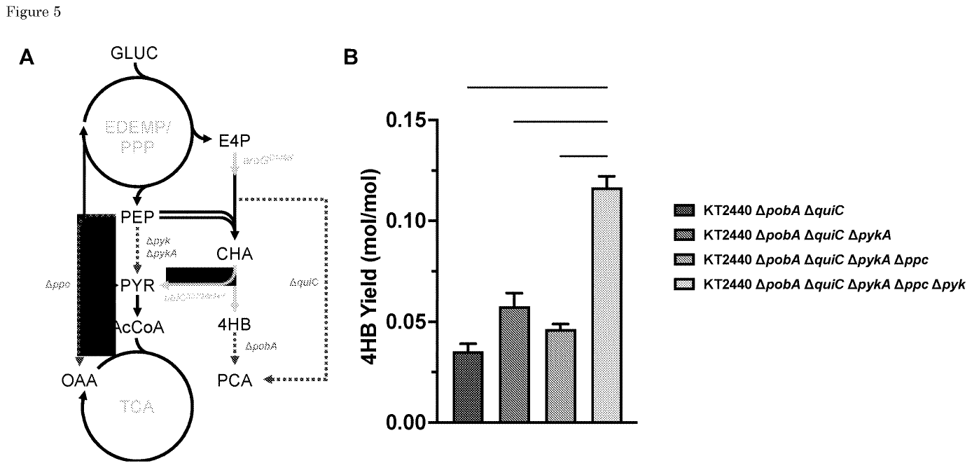

After demonstrating the successful conversion of 4HB to phenol and anisole, an aim was set to establish 4HB production from glucose. The production of 4HB is catalyzed by the enzyme chorismate pyruvate lyase, which cleaves chorismate equimolarly into 4HB and pyruvate. To establish 4HB production, the feedback-resistant gene (ubiC^{E31Q/M34V}) was taken from E. coli (Jha et al., 2019. ACS Synth Biol 8: 775-786). To push the flux in the shikimate pathway, the feedback-resistant aroG^{D146N} gene from E. coli was added and both genes were cloned on a pSEVAb23 vector. To further enhance the flux through the shikimate pathway, the quiC gene was deleted in KT2440 ApobA, which encodes a 3-dehydroshikimate (DHS) dehydratase (FIG. 5A). This enzyme converts the shikimate pathway intermediate 3DHS to PCA, diverting the flux away from 4HB production. This strain was grown with 50 mM glucose and 4HB production was measured. Although produced, the 4HB yield was rather low (0.035 mol 4HB/mol glucose) (FIG. 5B). It was hypothesized that there might be a precursor limitation. The shikimate pathway requires one mole of erythrose-4-phosphate (E4P) and two moles of phosphoenolpyruvate (PEP) to synthesize chorismate, the final product and precursor to 4HB. To increase PEP availability, the gene pykA was firstly deleted. This gene encodes a pyruvate kinase, which cleaves PEP to pyruvate (FIG. 5A). This was deemed effective as a yield of 0.057 mol/mol was measured, an increase of 63.4%. To further increase the PEP pool, the ppc gene encoding a phosphoenolpyruvate carboxylase, which converts PEP into oxaloacetate, was deleted. However, this deletion rather reduced the yield. At last, the gene pyk, the second isoenzyme for pyruvate kinase, was deleted. This deletion completely decouples all metabolic nodes from PEP, leaving the shikimate pathway as the sole entry point. As expected, this was deemed the most effective, yielding 0.116 mol/mol, an increase of 232.6%, compared to KT2440 ApobA AquiC strain.

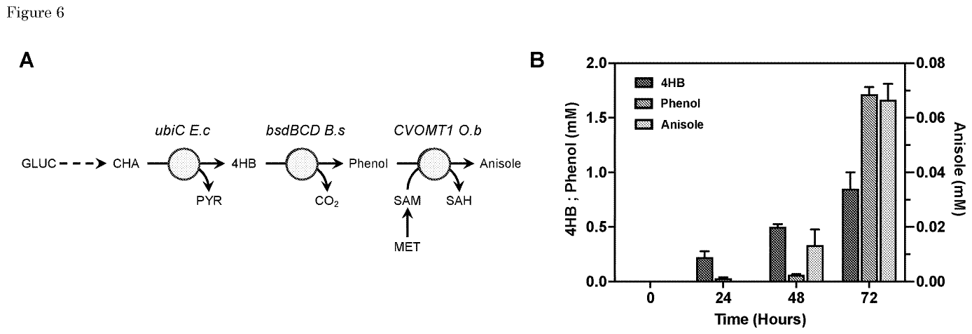

So far, it was demonstrated that anisole can be produced from 4HB as a precursor. In addition, 4HB production from glucose was established. Therefore, an aim was set to connect these two modules and produce anisole from glucose as the sole carbon source. Therefore, the aroG^{D146N} and ubiC^{E31Q/M34V} genes from E. coli, the BscBCD operon from B. subtilis and the cvomt1 gene from O. basilicum were combined on a pSEVAb23 vector (FIG. 6A). For the production experiment, the highest 4HB-producing strain KT2440 ApobAAquiCApykApykAAppc was selected. This strain was equipped with the anisole production plasmid and it was grown in minimal media containing 50 mM glucose and 2 mM methionine to increase the intracellular SAM pool. This strain produced 0.067 mM anisole, showcasing biological anisole production from glucose as a feedstock (FIG. 6B). However, proportional quantities of 4HB and phenol were still detected, 0.85 mM and 1.72 mM, respectively. This indicates that both the 4HB decarboxylase and the CVOMT1 enzyme are still bottlenecks in biological anisole production from glucose. It was hypothesized that the feedback-resistant UbiC generates 4HB at a faster rate than the 4HB decarboxylase can convert it, allowing 4HB accumulation. Therefore, promoters of both reactions should be finetuned to allow a smooth conversion from glucose into phenol. Phenol is most likely accumulated because it is a poor substrate for CVOMT1, and the enzyme needs to be subjected to further optimization to enhance its substrate specificity towards phenol.

### Developing a Growth-Coupled Selection System

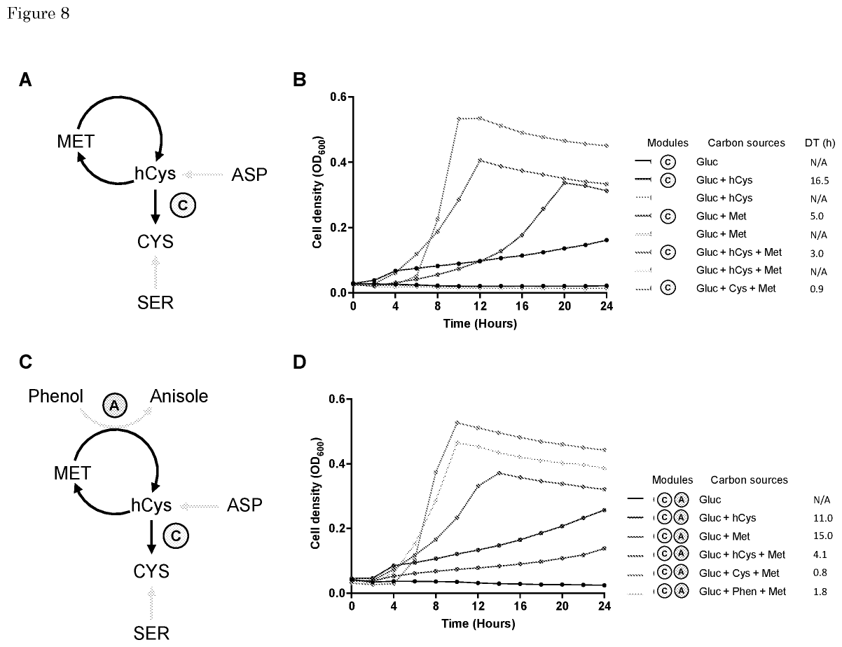

To optimize the substrate specificity of CVOMT1 towards phenol, a selection system was used in which growth is coupled to SAM methylation (Luo et al., 2019. PLoS Biol 17: 1-13). This design relies on the conversion of methionine into cysteine through the intermediate homocysteine with the aid of a SAM-dependent methyltransferase. Exogenously added methionine is converted into SAM, which would subsequently be converted into S-adenosyl-l-homocysteine (SAH) by CVOMT1 (FIG. 8C). Then, SAH is converted into homocysteine and subsequently into cysteine by reverse trans-sulfuration restoring growth in the process. Thus, the better the kinetics of CVOMT1 towards phenol, the faster growth would be restored.

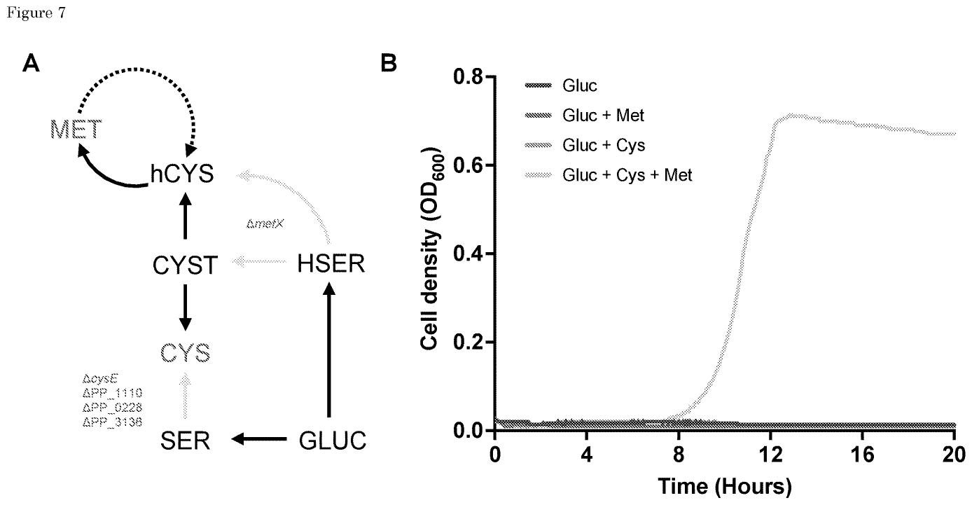

For this design, an aim was set to construct a double auxotrophic strain for the amino acids cysteine and methionine (FIG. 7A). Cysteine biosynthesis is derived from serine and is catalyzed by serine O-acetyltransferases. According to the KEGG database, this reaction is encoded by the genes cysE, PP_1110, PP_0228, and PP_3136, of which the latter two are putative. First, we examined the effect of only removing the annotated cysE and PP_1110 genes was examined. However, this strain was still able to grow in minimal media with glucose, indicating a flux towards cysteine biosynthesis (data not shown)). Next, the PP_0228 and PP_3136 genes were individually deleted from this strain to test whether these putative genes are involved in cysteine biosynthesis. The deletion of PP_3136 had no effect, and growth in minimal media with glucose could still occur (data not shown). However, the deletion of PP_0228 rendered the strain unable to synthesize cysteine, making it dependent on supplementation (data not shown). This would indicate that the PP_3136 gene is misannotated, not expressed, or involved in cysteine biosynthesis but not able to sustain enough flux to rescue growth. Nonetheless, PP_3136 was deleted to ensure a tight selection and the module was completely decoupled from the main metabolism. Apart from being derived from serine, cysteine can be produced through the trans-sulfuration of cystathionine, an intermediate within methionine biosynthesis. In theory, cysteine could still be produced through this pathway in P. putida, yet it was observed that the intracellular flux cannot rescue growth (data not shown). To completely uncouple methionine and cysteine biosynthesis from the central metabolism the metX gene, encoding a homoserine O-acetyltransferase, was deleted. The growth of this strain, termed CM-Aux, could only be restored through the addition of both cysteine and methionine, indicating a tight selection system (FIG. 7B).

### Testing the Synthetic Modules

Next, an aim was set to install the reverse trans-sulfuration module which will convert homocysteine to the amino acid cysteine. This is a two-step process, catalyzed by the enzymes cystathionine-β-synthase and cystathionine-γ-lyase. The C-module was created by cloning the cystathionine-β-synthase (Cys) from S. cerevisiae and the cystathionine-γ-lyase (PA) from Pseudomonas aeruginosa on a pSEVAb62 backbone. The CM-Aux strain was equipped with the C-module and growth of the strain in minimal media was assessed. In all cases, growth could only occur upon the expression of the C-module. This module converts homocysteine to methionine, and if active, produces cysteine, restoring growth (FIG. 8A). The addition of homocysteine only led to minor cell growth with a doubling time of 16.5 hours. When methionine was supplied together with homocysteine, growth was improved to a doubling time of 3 hours. As methionine is externally applied in this scenario, all homocysteine can be converted towards cysteine, most likely supporting higher growth rates. Interestingly, also with methionine as the sole supplementation, growth could occur with a doubling time of 5 hours. This indicates that the native methyltransferases of P. putida have sufficient activity to restore growth. Although this side activity renders the selection less tight, cells with kinetically fast CVOMT1 enzymes should theoretically still grow faster. To validate this hypothesis, the A-Module (the native CVOMT1 enzyme) was implemented together with the C-module in CM-Aux (FIG. 8C). It was observed that with both modules, the growth with homocysteine was improved (doubling time 11 hours), yet growth with methionine as sole supplementation was reduced to a doubling time of 15 hours. However, upon the addition of phenol and methionine, a doubling time of 1.8 hours was observed (FIG. 8D). In this scenario, the CVOMT1 enzyme converts phenol and SAM to anisole and SAH. Then the SAH is converted into homocysteine and subsequently to cysteine, restoring growth. It was observed that the CVOMT1 enzyme is active and that its activity can be coupled with growth (FIG. 8D; purple line).

### Evolution of CVOMT1

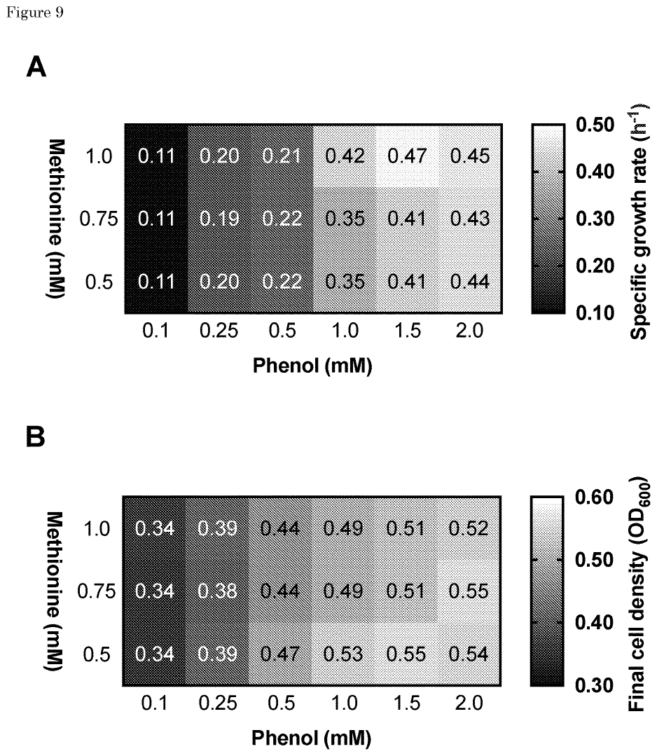

Having established a growth-coupled selection strategy, an aim was set to evolve kinetically better CVOMT1 enzymes. In the current selection system, growth with CVOMT1 is relatively fast compared with the control (doubling time of 1.8 and 0.8 hours, respectively) (FIG. 8D). Here, 1 mM of methionine and phenol were supplied which most likely saturates the enzyme, thereby favouring kinetics and limiting the selection. The methionine and phenol concentrations were varied to find the optimal conditions to select kinetically better CVOMT1 enzymes (FIG. 9). Methionine supplies SAM and its availability did not seem to hamper enzyme kinetics and therefore growth. This is in accordance with the literature, as CVOMT1 naturally has a Michaelis constant (Km) of 6 nM for SAM (Gang et al., 2002. Plant Cell 14: 505-519). Therefore, the main limitation is phenol which is a poor substrate for CVOMT1. It was observed that with increasing phenol concentrations, growth accelerates, as phenol is more rapidly converted and can supply the cell with cysteine for growth (FIG. 9A). The less phenol is added, the less cysteine will be produced, which is reflected in the final observed cell density (FIG. 9B). For biological anisole production, fast enzymes are needed with a high affinity towards phenol and therefore a low Km. This is especially important as a high Km would require the build-up of intracellular phenol which might hamper bioproduction. Therefore, to screen potential better candidates, a phenol concentration of 0.1 mM was selected.

The optimization of CVOMT could be performed through either random mutagenesis PCR or adaptive laboratory evolution. Although both are efficient, in the latter one there is the possibility that native methyltransferases will be able to rescue growth, a phenomenon that has been observed by Luo et al., 2019 (Luo et al., 2019. PLoS Biol 17: 1-13). In their study, they actively improved two methyltransferases regarding melatonin biosynthesis, yet noticed the contribution of native methyltransferases to bacterial growth. The inventors also observed minor growth using native methyl-transferases when methionine was the sole supplementation (FIG. 8D, green line). Therefore, a randomized approach was used using error-prone PCR on the cvomt1 gene from O. basilicum. This library was transformed into CM-Aux containing the C-module and grown overnight in minimal media with 10 mM glucose, 1 mM methionine, and 0.1 mM phenol to reduce the population that harbors kinetically slower enzymes. Hereafter, the culture was plated on minimal agar with the same carbon sources. In total, 92 visible large variants were selected, and their growth was compared to the wild-type CVOMT1 enzyme. It was observed that all variants grew significantly faster compared to the wild-type enzyme (data not shown). However, the growth rates between the isolates were not as diverse as expected. Therefore, the phenol concentration was reduced by ten-fold to 0.01 mM to further tighten the selection system and screen for variants with high selectivity towards phenol. The reduction of phenol proved to be effective, as the growth rates of the isolates were more diverse (data not shown).

### Evaluation of Isolated Mutants

The cvomt1 gene of the five fastest-growing isolates were sequenced and it was discovered that all genes had the same mutation in which aspartic acid (Asp) at position 264 was substituted with asparagine (Asn). According to Gang et al., 2002 (Gang et al., 2002. Plant Cell 14: 505-519), the aminoacidic residues His-263, Asp-264 and Glu-322 in CVOMT1 are implicated in catalysis. Asparagine is the amide form of aspartic acid and has a high propensity to form hydrogen bonds (Vennelakanti et al., 2021. Chem Sci 12: 1147-1162). The three-dimensional structure of the evolved enzyme reveals that the replaced Asn-264 is now able to form hydrogen bonds with Met-314, causing a conformational change of Phe-310 (data not shown). Both Phe-310 and Met-314 are involved in substrate binding of the isoflavone O-methyltransferase of Medicago sativa, which is distantly related to cvomt1 from O. basilicum (Gang et al., 2002. Plant Cell 14: 505-519). Therefore, it could be possible that this conformational change allows an increased binding affinity towards phenol. Moreover, it is likely that Asn-264 supports the stabilization of phenol in the active site. Hydrogen bonds are generally considered to play a key role in protein-ligand binding and enzyme catalysis Chen et al., 2016. Sci Adv 2: 1126/sciadv.1501240). In CVOMT1, Asp-264 is less favorable to making hydrogen bonds and substrate stabilization is performed by His-263 alone (Gang et al., 2002. Plant Cell 14: 505-519). However, to validate the biochemical reason for the enhanced enzyme kinetics, further research would be required.

### Anisole In-Situ Extraction

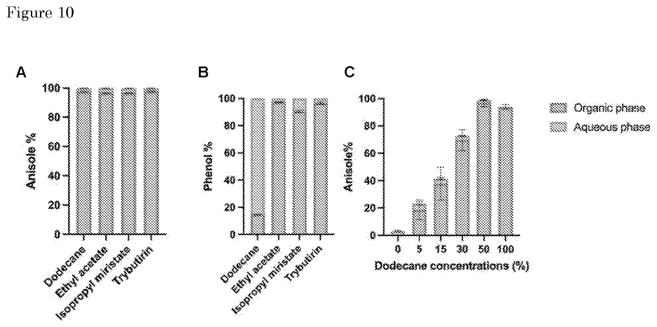

A main bottleneck when producing anisole is its volatility. To overcome this problem an organic solvent can be used in biphasic fermentation to quantify volatile or insoluble products in water, allowing their recovery. To enable in situ anisole extraction, we tested four organic solvents: dodecane, ethyl acetate, isopropyl myristate, and tributyrin. For this experiment, we prepared a stock solution of 1 mM of phenol and 1 mM of anisole dissolved in water. Next, 0.9 ml of the stock solution was mixed with an equal volume of one of the selected solvents in an Eppendorf tube. After 1 hour of incubation, the different solutions were centrifuged to separate the two phases and the phenol and anisole concentrations were quantified for each phase by HPLC. All four tested organic solvents successfully captured anisole (FIG. 10A).

In addition, the phenol extraction capacity of the organic solvents was evaluated. Phenol is the precursor of anisole and is supplied to the cultures when screening for new potential phenol methyltransferases or optimizing the current enzymes available. Therefore, we opted for an organic solvent that could extract anisole, but not phenol. Of all organic phases tested, except in dodecane, phenol was mostly detected in the organic phase (FIG. 10B). Dodecane extracted only 14.82% of the detected phenol, while the majority of this compound retained in water. Therefore, dodecane was chosen as the solvent for anisole extraction.

An additional experiment was performed to determine the optimal ratio of the two phases. Ratios of 0%, 5%, 15%, 30%, 50%, and 100% dodecane were tested in a total volume of 25 ml. First, 1 mM of anisole and 1 mM of phenol were dissolved in water and distributed among different flasks. Next, dodecane was added to the flasks according to the predetermined ratios. Flasks were incubated for 24 h at 30° C. and 250 rpm to simulate P. putida KT2440 growth. After incubation, samples were collected separately from each phase and concentrations were measured using HPLC. An evaporation of anisole was observed in all cases, but the amount evaporated decreased drastically with higher percentages of dodecane (FIG. 10C). After 24 hours, samples containing the organic phase from 5% and 15% dodecane flasks captured on average 19.42% and 39.57% of the initial concentration of anisole, respectively. In the water phase (0% dodecane), anisole was almost undetectable. Flasks containing 30% dodecane, extracted approximately 75% of the anisole which was further increased to nearly 95% flasks with a 50% ratio.

### Characterization of POMT1

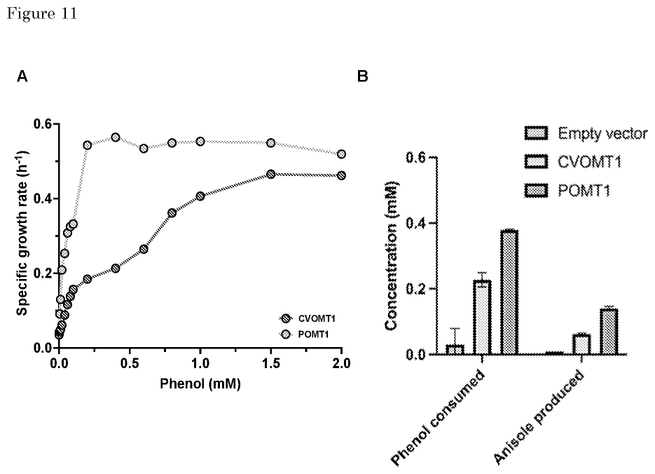

The newly evolved variant was rebranded as POMT1 (phenol O-methyltransferase) and its substrate-binding affinities were analyzed in CM-Aux using 1 mM of methionine and different phenol concentrations (FIG. 11A). POMT1 outcompetes CVOMT1 on all supplied phenol concentrations, reaching its maximum growth rate at a concentration of 0.2 mM, highlighting its superior kinetics. Next, an aim was set to examine the anisole production rate of POMT1 compared to CVOMT1. P. putida KT2440 was transformed with either candidate and the strains were inoculated in M9 media and fed with 50 mM glucose, 1 mM phenol and 1 mM Methionine. An initial sample was taken and hereafter 50% dodecane was added. Cells were then incubated overnight at 30° C. and 250 rpm. Samples were taken at time zero only from the medium and then after 24 hours from both phases and analyzed at the HPLC.

Over the course of 24 hours, CVOMT1 consumed 0.23 mM phenol (FIG. 11B). POMT1 consumed 65% more within 24 hours, with a total of 0.38 mM. This finding suggests a stronger substrate affinity towards phenol of the latter enzyme. Both candidates produced anisole throughout the experiment, with CVOMT1 at a maximum of 0.06 mM and POMT1 at a maximum of 0.14 mM (FIG. 11B). Although not all the phenol was converted into anisole, the 2.33-fold increase in anisole concentration in the POMT1 replicates confirms its enhanced enzymatic activity compared to the wild-type enzyme.

Sequences

SEQ ID NO: 1 Sequence of cvomt1 of Ocimum basilicum, having D264
MALQNMDISL STEQLLQAQA HVWNHMYAFA NSMSLKCAIQ LGIPDILHKH
DHPMTLSQLL KAIPINKEKS QSFQRLMRAL VNSNFFIEEN SNNQEVCYWL
TPASRLLLKG APLTVAPLVQ VVLDPTFTNP WHYMSEWFKH ENHATQFEAA
NGCTFWEKLA NKPSMGRFFD EAMSCDSRLV AHVLTKDYKH VIDGIRTLVD
VGGGNGTMAK AIVEAVPTMK CTVLDLPHVV AGLESTDKLS YIGGDMFQSI
PSADAILLKF IIHDWDDEEG LKILKRCKDA VGIGGKVIII DVVVGVNHDV
DEVLEDQLHF DMAMMSYFNA KERTMNEWEK LISAAGFTSY KLTPAFGVRS
LIEAYP
SEQ ID NO: 2. Codon-optimized coding sequence of cvomt1 of Ocimum
basilicum
Atggccctgcagaacatggacatctcgctgtcgaccgaacagctgctgcaggcccaggcccacg
tgtggaaccacatgtacgccttcgccaactcgatgtcgctgaagtgcgccatccagctgggcat
cccggacatcctgcacaagcacgaccacccgatgaccctgtcgcagctgctgaaggccatcccg
atcaacaaggaaaagtcgcagtcgttccagcgcctgatgcgcgccctggtgaactcgaacttct
tcatcgaagaaaactcgaacaaccaggaagtgtgctactggctgaccccggcctcgcgcctgct
gctgaagggcgccccgctgaccgtggccccgctggtgcaggtggtgctggacccgaccttcacc
aacccgtggcactacatgtcggaatggttcaagcacgaaaaccacgccacccagttcgaagccg
ccaacggctgcaccttctgggaaaagctggccaacaagccgtcgatgggccgcttcttcgacga
agccatgtcgtgcgactcgcgcctggtggcccacgtgctgaccaaggactacaagcacgtgatc
gacggcatccgcaccctggtggacgtgggcggcggcaacggcaccatggccaaggccatcgtgg
aagccgtgccgaccatgaagtgcaccgtgctggacctgccgcacgtggtggccggcctggaatc
gaccgacaagctgtcgtacatcggcggcgacatgttccagtcgatcccgtcggccgacgccatc
ctgctgaagttcatcatccacgactgggacgacgaagaaggcctgaagatcctgaagcgctgca
aggacgccgtgggcatcggcggcaaggtgatcatcatcgacgtggtggtgggcgtgaaccacga
cgtggacgaagtgctggaagaccagctgcacttcgacatggccatgatgtcgtacttcaacgcc
aaggaacgcaccatgaacgaatgggaaaagctgatctcggccgccggcttcacctcgtacaagc
tgaccccggccttcggcgtgcgctcgctgatcgaagcctacccgtaa
SEQ ID NO: 3 Sequence of POMT1 (phenol O-methyltransferase 1)
MALQNMDISL STEQLLQAQA HVWNHMYAFA NSMSLKCAIQ LGIPDILHKH
DHPMTLSQLL KAIPINKEKS QSFQRLMRAL VNSNFFIEEN SNNQEVCYWL
TPASRLLLKG APLTVAPLVQ VVLDPTFTNP WHYMSEWFKH ENHATQFEAA
NGCTFWEKLA NKPSMGRFFD EAMSCDSRLV AHVLTKDYKH VIDGIRTLVD
VGGGNGTMAK AIVEAVPTMK CTVLDLPHVV AGLESTDKLS YIGGDMFQSI
PSADAILLKF IIHNWDDEEG LKILKRCKDA VGIGGKVIII DVVVGVNHDV
DEVLEDQLHF DMAMMSYFNA KERTMNEWEK LISAAGFTSY KLTPAFGVRS
LIEAYP
SEQ ID NO: 4 UbiC from E. coli (UniProt P26602)
MSHPALTQLR ALRYCKEIPA LDPQLLDWLL LEDSMTKRFE QQGKTVSVTM
IREGFVEQNE IPEELPLLPK ESRYWLREIL LCADGEPWLA GRTVVPVSTL
SGPELALQKL GKTPLGRYLF TSSTLTRDFI EIGRDAGLWG RRSRLRLSGK
PLLLTELFLP ASPLY
SEQ ID NO: 5 4-hydroxybenzoate (4HB) decarboxylase from Bacillus
subtilis
MAYQDFREFLAALEKEGQLLTVNEEVKPEPDLGASARAASNLGDKSPALLFNNIYGYHNARIAM
NVIGSWPNHAMMLGMPKDTPVKEQFFEFAKRYDQFPMPVKREETAPFHENEITEDINLFDILPL
FRINQGDGGYYLDKACVISRDLEDPDNFGKQNVGIYRMQVKGKDRLGIQPVPQHDIAIHLRQAE
ERGINLPVTIALGCEPVITTAASTPLLYDQSEYEMAGAIQGEPYRIVKSKLSDLDVPWGAEVVL
EGEIIAGEREYEGPFGEFTGHYSGGRSMPIIKIKRVYHRNNPIFEHLYLGMPWTECDYMIGINT
CVPLYQQLKEAYPNEIVAVNAMYTHGLIAIVSTKTRYGGFAKAVGMRALTTPHGLGYCKMVIVV
DEDVDPFNLPQVMWALSTKMHPKHDAVIIPDLSVLPLDPGSNPSGITHKMILDATTPVAPETRG
HYSQPLDSPLTTKEWEQKLMDLMNK
SEQ ID NO: 6 aroG from E. coli (UniProt POAB91)
MNYQNDDLRIKEIKELLPPVALLEKFPATENAANTVAHARKAIHKILKGNDDRLLVVIGPCSIH
DPVAAKEYATRLLALREELKDELEIVMRVYFEKPRTTVGWKGLINDPHMDNSFQINDGLRIARK
LLLDINDSGLPAAGEFLDMITPQYLADLMSWGAIGARTTESQVHRELASGLSCPVGFKNGTDGT
IKVAIDAINAAGAPHCFLSVTKWGHSAIVNTSGNGDCHIILRGGKEPNYSAKHVAEVKEGLNKA
GLPAQVMIDFSHANSSKQFKKQMDVCADVCQQIAGGEKAIIGVMVESHLVEGNOSLESGEPLAY
GKSITDACIGWEDTDALLRQLANAVKARRG
SEQ ID NO: 7 pobA gene of Pseudomonas putida (UniProt Q9R9T1)
MKTQVAIIGAGPSGLLLGQLLHNAGIETVIVERQTPEYVLGRIRAGVLEQGTVDLLREAGVSAR
MDREGLVHEGVELLVGGRRQRLDLKALTGGKTVMVYGQTEVTRDLMQAREASGAPIIYAANNVQ
PHELKGERPYLTFEKDGQAHRLECDYIAGCDGFHGVSRQSIPEGVLKQYERVYPFGWLGLLSDT
PPVNHELIYAHHERGFALCSQRSQTRSRYYLQVPLDDKVEAWSDERFWDELKARLPAEVAADLV
TGPALEKSIAPLRSLVVEPMQYGHLFLVGDAAHIVPPTGAKGLNLAASDVNYLYRILVKVYGEG
RTDLLQQYSPLALRRVWKGERFSWFMTQLLHDFGSHKDAWDQKMQEADREYFLNSPAGLLNIAE
NYVGLPYEAVV

TABLE 1
Strains

| | | |
|---|---|---|
| Strain | Description | Source |

Escherichia coli

| | | |
|---|---|---|
| Dh5a | Cloning host: F-λ-endA1 glnX44(AS) thiE1 recA1 relA1 | Grant et al., 1990. Genetics |
| | spoT1 gyrA96(NalR) rfbC1 deoR nupG Φ80(lacZΔM15) Δ(argF-lac)U169 | 87: 4645-4649 |
| | hsdR17(rK mK) | |
| CC118λpir | Cloning host: araD139 Δ(ara-leu) 7697 ΔlacX74 galE galK phoA20 thi- 1 | Herrero et al., 1990. J |
| | rpsE rpoB(RifR) argE(Am) recA1, λpir lysogen | Bacteriol 172: 6557-6567 |

Pseudomonas putida

| | | |
|---|---|---|
| KT2440 | Wild-type strain; mt-2 derivative cured of the TOL plasmid pWW0 | Bagdasarian et al., 1981. |
| | | Gene 16: 237-247 |
| KT2440 ΔpobA | KT2440 with scarless deletion of the pobA gene | This study |
| KT2440 ΔpobA ΔquiC | KT2440 with scarless deletion of the pobA and quiC genes | This study |
| KT2440 ΔpobA ΔquiC | KT2440 with scarless deletion of the pobA, quiC, and pykA genes | This study |
| ΔpykA | | |
| KT2440 ΔpobA ΔquiC | KT2440 with scarless deletion of the pobA, quiC,, pykA and ppc genes | This study |
| ΔpykA Δppc | | |
| KT2440 ΔpobA ΔquiC Δpyk | KT2440 with scarless deletion of the pobA, quiC, pyk, pykA and ppc | This study |
| ΔpykA Δppc | genes | |
| KT2440 ΔcysE ΔPP_1110 | KT2440 with scarless deletion of the cysE, and PP_1110 genes | This study |
| KT2440 ΔcysE ΔPP_1110 | KT2440 with scarless deletion of the cysE, PP_1110 and PP_0228 genes | This study |
| ΔPP_0228 | | |
| KT2440 ΔcysE ΔPP_1110 | KT2440 with scarless deletion of the cysE, PP_1110 and PP_3136 genes | This study |
| ΔPP_3136 | | |
| KT2440 ΔcysE ΔPP_1110 | KT2440 with scarless deletion of the cysE, PP_1110, PP_0228 and | This study |
| ΔPP_0228 ΔPP_3136 | PP_3136 genes | |
| CM - Aux | KT2440 with scarless deletion of the cysE, PP_1110, PP_0228, PP_3136 | This study |
| | and metX genes | |

TABLE 2
plasmids

| Plasmid | Description | Source |
|---|---|---|
| pGNW | Plasmid for genome editing in Gram-negative bacteria: ori (R6K) neo, KmR | Wirth et al., 2020. Microb |
| | | Biotechnol 12: 233-249 |
| pGNW - pyk | pGNW for scarless deletion of pyk | This study |
| pGNW - | pGNW for scarless deletion of pykA | This study |
| pykA | | |
| pGNW - ppc | pGNW for scarless deletion of ppc | This study |
| pGNW - | pGNW for scarless deletion of pobA | This study |
| pobA | | |
| pGNW - | pGNW for scarless deletion of quiC | This study |
| quiC | | |
| pGNW - cysE | pGNW for scarless deletion of cysE | This study |
| pGNW - | pGNW for scarless deletion of PP_1110 | This study |
| PP_1110 | | |
| pGNW - | pGNW for scarless deletion of PP_0228 | This study |
| PP_0228 | | |
| pGNW - | pGNW for scarless deletion of PP_3136 | This study |
| PP_3136 | | |
| pGNW- | pGNW for scarless deletion of metX | This study |
| metX | | |
| pGNW- | pGNW containing two lox sites and the bsdBCD operon from Bacillus subtilis | This study |
| Phenol | | |
| pGNW- | pGNW containing two lox sites, the T7 RNA polymerase and the Cre recombinase | This study |
| Landing pad | under the control of the t7 promoter. | |
| pQURE6-H | Helper plasmid; oriV(RK2), xylS, PmI-SceI; GmR | Volke et al., 2020. Metab Eng |
| | | Commun 10: e00126 |
| pSB1C3 | Repository vector, pUC19-derived pMB1 ori, Cm^{R} | IGEM registry |
| pSB1C3_CVOMT | pSB1C3 with the codon optimized cvomt1 gene from Ocimum basilicum | This study |
| pSEVAb23 | oriV(pBBR1); KM^{R} | Damalas et al., 2020. Microb |
| | | Biotechnol 13: 1793-1806 |
| pC | pSEVAb62 with the cys4 from Saccharomyces cerevisiae and PA_0400 from | This study |
| | Pseudomonas aeruginosa. | |
| pA | pSEVAb23 with the codon optimized cvomt1 gene from Ocimum basilicum | This study |
| pPOMT | pSEVAb23 with the comt1 gene from Ocimum basilicum containing the | This study |
| | D264N mutation | |
| p4HB | pSEVAb23 with the aroG^{D146N} and ubiC^{E31Q/M34V} from E. coli | This study |
| P4A | pSEVAb23 with the bsdBCD operon from Bacillus subtilis and cvomt1 gene | This study |
| | from Ocimum basilicum | |
| pAnisole | pSEVAb23 with the aroG^{D146N} and ubiC^{E31Q/M34V} from E. coli, the bsdBCD | This study |
| | operon from Bacillus subtilis and cvomt1 gene from Ocimum basilicum | |

TABLE 3
primers

| Primers | Sequence ′5 > 3′ | Purpose |
|---|---|---|
| HA1_pykA_ | aggtctctcccgttttccagggtctgcaggcg | Cloning the |
| FW | | homology |
| HA1_pykA_ | aggtctctgcacagactcctatagtgaagc | arms for |
| RV | | scarless |
| HA2_pykA_ | aggtctctgtgctccactcgtttcacagcacaagg | deletion of |
| FW | | pykA |
| HA2_pykA_ | aggtctcttcgacctggcagccatcatcgacc | |
| RV | | |
| HA1_pyk_ | aggtctctcccgaggctgccgcggtcaaagc | Cloning the |
| FW | | homology |
| HA1_pyk_ | aggtctcttcatgattgggcagtctcaagg | arms for |
| RV | | scarless |
| HA2_pyk_ | aggtctctatgagattgccgggggcgcg | deletion of |
| FW | | pyk |
| HA2_pyk_ | aggtctcttcgacaatatggccgaccagggcag | |
| RV | | |
| HA1_ppc_ | aggtctctcccgtgctcaagttgtgcaggtgctg | Cloning the |
| FW | | homology |
| HA1_ppc_ | aggtctcttggcgctctctccgttgc | arms for |
| RV | | scarless |
| HA2_ppc_ | aggtctctgccagggcaagcccgggtcatg | deletion of |
| FW | | ppc |
| HA2_ppc_ | aggtctcttcgacagctggccggcatccatg | |
| RV | | |
| HA1_pobA_ | aggtctctcccgcaggttcgctacaagggc | Cloning the |
| FW | | homology |
| HA1_pobA_ | aggtctcttgttgttgtctctgttcggacg | arms for |
| RV | | scarless |
| HA2_pobA_ | aggtctctaacagggctgcgagctacg | deletion of |
| FW | | pobA |
| HA2_pobA_ | aggtctcttcgatcctccttggggagcatttt | |
| RV | | |
| HA1_quiC_ | aggtctctcccgcgaaggcgccgatcgggc | Cloning the |
| FW | | homology |
| HA1_quiC_ | aggtctctggcgggagttccttgttgtt | arms for |
| RV | | scarless |
| HA2_quiC_ | aggtctctcgcctcgcctgcaccggcctgtc | deletion of |
| FW | | quiC |
| HA2_quiC_ | aggtctcttcgacacgctggtggatgcagcgc | |
| RV | | |
| HA1_cysE_ | aggtctctcccgcctggccgttgatcggcccg | Cloning the |
| FW | | homology |
| HA1_cysE_ | aggtctctctgtcagtccttccgtttatgc | arms for |
| RV | | scarless |
| HA2_cysE_ | aggtctctacagtcgcgccgcactgaagtcatgg | deletion of |
| FW | | cysE |
| HA2_cysE_ | aggtctcttcgacgtgggtcaggcaggtatcacc | |
| RV | | |
| HA1_PP_1110_ | aggtctctcccgctgggggcagccagccgctg | Cloning the |
| FW | | homology |
| HA1_PP_1110_ | aggtctcttgggtgcatgggcttgatatcc | arms for |
| RV | | scarless |
| HA2_PP_1110_ | aggtctctcccaatgaacgctctgaactacgaag | deletion of |
| FW | | PP_1110 |
| HA2_PP_1110_ | aggtctcttcgactcgaccaggaagtcgaaacgtg | |
| RV | | |
| HA1_PP_0228_ | aggtctctcccgttcgaagccggtcagtttgccg | Cloning the |
| FW | | homology |
| HA1_PP_0228_ | aggtctctggttttctcctgccgtataggc | arms for |
| RV | | scarless |
| HA2_PP_0228_ | aggtctctaacctcccgcagtgcaattgcctgtgc | deletion of |
| FW | | PP_0228 |
| HA2_PP_0228_ | aggtctcttcgacagacaaggcggtgaccaccacg | |
| RV | | |
| HA1_PP_3136_ | aggtctctcccgatggtcgacgacatgaactggg | Cloning the |
| FW | | homology |
| HA1_PP_3136_ | aggtctctggctccacctaccttgtcgg | arms for |
| RV | | scarless |
| HA2_PP_3136_ | aggtctctagccggcctatggcaacggtgatcg | deletion of |
| FW | | PP_3136 |
| HA2_PP_3136_ | aggtctcttcgacgaagccacccacggcagcaaagg | |
| RV | | |
| HA1_metX_ | aggtctctcccgggctggatacctcgtcgctactgc | Cloning the |
| FW | | homology |
| HA1_metX_ | aggtctctcgaccctgctcacgcttgacgg | arms for |
| RV | | scarless |
| HA2_metX_ | aggtctctgtcgttgccatctgaggacagcatgag | deletion of |
| FW | | metX |
| HA2_metX_ | aggtctcttcgacgcccggcgttcgtgcacaagttc | |
| RV | | |
| CVOMT_ALE_ | aggtctctcatcctgctgaagagcatcatccacgactgg | Generating a |
| FW | | library of |
| CVOMT_ALE_ | aggtctctgatggcgtcggccgacgggatcg | kinetically |
| FW | | better |
| | | CVOMT1 |
| | | enzymes. |
| CYS4_ | aggtctctactagagaaagaggagaaatactagatgactaaatct | Amplification |
| FW | gagcagcaagccg | of cys4 from |
| CYS4_ | aggtctctgggcgtattattatgctaagtagctcagtaaatcc | S. cerevisiae |
| RV | | and PA_0400 |
| PA_0400_ | aggtctctgcccgaaagaggagaaatactagatgagccagcacga | from P. |
| FW | ccagcatcc | aeruginosa |
| PA_0400_ | taaggtctcgactgcagcggccgctactagtattattagatcttcgc | for the |
| RV | cagcgcctgcgcc | construction |
| | | of the C- |
| | | module. |
| bsdB_ | tggtctcaccaagaaagaggagaaatactagatgaaagcagaat | Amplification |
| FW | tcaagcgtaaagg | of the |
| bsdB_ | aggtctctggatgtattattaagctcctcctttttgtttttcaatccc | bsdBCD |
| RV | | genes from |
| bsdC_ | tggtctcaatccgaaagaggagaaatactagatggcttatcaaga | B. subtilis |
| FW | tttcagagaatttc | encoding the |
| bsdC_ | aggtctcttaccgtattattatttattcattaagtccattagtttttg | 4HB |
| RV | | decarboxylase. |
| bsdD_ | tggtctcaggtagaaagaggagaaatactagatgcatacatgtcc | |
| FW | tcgatgcg | |
| bsdD_ | taaggtctcgactgcagcggccgctactagtattattaagcctttcgt | |
| RV | tccggcacc | |
| aroG_ | aggtctctactagagaaagaggagaaatactagatgaattaccag | Amplification |
| FW | aacgatgatc | of |
| aroG_ | aggtctctgggcgtattattacccacgccgagctttcac | aroGD146N |
| RV | | |
| ubiC_ | aggtctctgcccgaaagaggagaaatactagatgtcacaccccgc | Amplification |
| FW | gttaac | of |
| ubiC_ | aggtctctttgggtattattagtacaacggtgacgcc | ubiCE31Q/M34V |
| RV | | |
| Loxpad_1 | aggtctctccagtgatcaacacctgactacccg | Construction |
| Loxpad_2 | aggtctctacaaactggttttagagaattaaaactg | of the |
| Loxpad_3 | aggtctcttagattattacgcgaacgcgaagtccgac | landing pad |
| Loxpad_4 | aggtctctatgaacacgattaacatcgctaag | for the |
| Loxpad_5 | aggtctctctggattctcaccaataaaaaacg | chromosomal |
| Loxpad_6 | aggtctctcttggactcctgttgatagatcc | integration |
| Loxpad_7 | aggtctcttcatctagtatttctcctctttctc | of the |
| Loxpad_8 | aggtctctggtactatagtgagtcgtattatggaattcgcggccgct | bsdBCD |
| | tctag | operon |
| Loxpad_9 | aggtctcttaccgttcgtatagcatacattatacg | |
| Loxpad_10 | aggtctctttgtgcagcccgatgccgaagagg | |
| Loxpad_11 | aggtctctcaagttataccagattgcgcagttcg | |
| Loxpad_12 | aggtctcttctagagttgacggctagctcag | |

1. A method for the production of anisole by a microorganism, comprising providing said microorganism with a phenol O-methyltransferase enzyme and a source of S-adenosyl-L-methionine;
incubating the microorganism in a growth medium in the presence of a phenol source;
to thereby produce anisole by the microorganism.

2. The method of claim 1, wherein the source of S-adenosyl-L-methionine is methionine.

3. The method of claim 1, whereby said phenol source is 4-hydroxybenzoate (4HB).

4. The method of claim 1, wherein the microorganism is further provided with a 4HB decarboxylase.

5. The method of claim 1, wherein the phenol source is a sugar, and the microorganism is further provided with a chorismate pyruvate lyase.

6. The method of claim 5, wherein the microorganism lacks functional expression of a 3-dehydroshikimate (DHS) dehydratase, a pyruvate kinase, a hydroxybenzoate hydroxylase, a phosphoenolpyruvate carboxylase, or a combination thereof.

7. The method of claim 5, wherein the chorismate pyruvate lyase is a feedback-resistant gene.

8. The method of claim 1, wherein the phenol O-methyltransferase enzyme has at least 70% sequence identity to a chavicol O-methyltransferase (EC 2.1.1.146).

9. The method of claim 1, wherein the phenol O-methyltransferase enzyme has at least 70% sequence identity to SEQ ID NO:1.

10. The method of claim 1, wherein an organic solvent is added to the growth medium to capture the produced anisole.

11. The method of claim 10, wherein the organic solvent comprises dodecane, ethyl acetate, or a mixture thereof.

12. The method of claim 1, wherein the microorganism is a bacterium or a yeast.

13. A phenol O-methyltransferase enzyme (POMT1) comprising the amino acid sequence of SEQ ID NO:1 comprising a D264N amino acid alteration.

14. A nucleic acid molecule that encodes the POMT1 of claim 13.

15. A microorganism comprising the nucleic acid molecule of claim 14.

16. (canceled)

17. (canceled)

18. (canceled)


SEQUENCE LISTING (75 sequences)

SEQ ID NO: 1 - protein, 356 aa, Ocimum basilicum
MALQNMDISLSTEQLLQAQAHVWNHMYAFANSMSLKCAIQLGIPDILHKHDHPMTLSQLLKAIPINKEKSQSFQRLMRALVNSNFFIEENSNNQEVCYWLTPASRLLLKGAPLTVAPLVQVVLDPTFTNPWHYMSEWFKHENHATQFEAANGCTFWEKLANKPSMGRFFDEAMSCDSRLVAHVLTKDYKHVIDGIRTLVDVGGGNGTMAKAIVEAVPTMKCTVLDLPHVVAGLESTDKLSYIGGDMFQSIPSADAILLKFIIHDWDDEEGLKILKRCKDAVGIGGKVIIIDVVVGVNHDVDEVLEDQLHFDMAMMSYFNAKERTMNEWEKLISAAGFTSYKLTPAFGVRSLIEAYP

SEQ ID NO: 2 - DNA, 1071 bp, synthetic construct
  source 1..1071: mol_type = other DNA
atggccctgcagaacatggacatctcgctgtcgaccgaacagctgctgcaggcccaggcccacgtgtggaaccacatgtacgccttcgccaactcgatgtcgctgaagtgcgccatccagctgggcatcccggacatcctgcacaagcacgaccacccgatgaccctgtcgcagctgctgaaggccatcccgatcaacaaggaaaagtcgcagtcgttccagcgcctgatgcgcgccctggtgaactcgaacttcttcatcgaagaaaactcgaacaaccaggaagtgtgctactggctgaccccggcctcgcgcctgctgctgaagggcgccccgctgaccgtggccccgctggtgcaggtggtgctggacccgaccttcaccaacccgtggcactacatgtcggaatggttcaagcacgaaaaccacgccacccagttcgaagccgccaacggctgcaccttctgggaaaagctggccaacaagccgtcgatgggccgcttcttcgacgaagccatgtcgtgcgactcgcgcctggtggcccacgtgctgaccaaggactacaagcacgtgatcgacggcatccgcaccctggtggacgtgggcggcggcaacggcaccatggccaaggccatcgtggaagccgtgccgaccatgaagtgcaccgtgctggacctgccgcacgtggtggccggcctggaatcgaccgacaagctgtcgtacatcggcggcgacatgttccagtcgatcccgtcggccgacgccatcctgctgaagttcatcatccacgactgggacgacgaagaaggcctgaagatcctgaagcgctgcaaggacgccgtgggcatcggcggcaaggtgatcatcatcgacgtggtggtgggcgtgaaccacgacgtggacgaagtgctggaagaccagctgcacttcgacatggccatgatgtcgtacttcaacgccaaggaacgcaccatgaacgaatgggaaaagctgatctcggccgccggcttcacctcgtacaagctgaccccggccttcggcgtgcgctcgctgatcgaagcctacccgtaa

SEQ ID NO: 3 - protein, 356 aa, synthetic construct
MALQNMDISLSTEQLLQAQAHVWNHMYAFANSMSLKCAIQLGIPDILHKHDHPMTLSQLLKAIPINKEKSQSFQRLMRALVNSNFFIEENSNNQEVCYWLTPASRLLLKGAPLTVAPLVQVVLDPTFTNPWHYMSEWFKHENHATQFEAANGCTFWEKLANKPSMGRFFDEAMSCDSRLVAHVLTKDYKHVIDGIRTLVDVGGGNGTMAKAIVEAVPTMKCTVLDLPHVVAGLESTDKLSYIGGDMFQSIPSADAILLKFIIHNWDDEEGLKILKRCKDAVGIGGKVIIIDVVVGVNHDVDEVLEDQLHFDMAMMSYFNAKERTMNEWEKLISAAGFTSYKLTPAFGVRSLIEAYP

SEQ ID NO: 4 - protein, 165 aa, Escherichia coli
MSHPALTQLRALRYCKEIPALDPQLLDWLLLEDSMTKRFEQQGKTVSVTMIREGFVEQNEIPEELPLLPKESRYWLREILLCADGEPWLAGRTVVPVSTLSGPELALQKLGKTPLGRYLFTSSTLTRDFIEIGRDAGLWGRRSRLRLSGKPLLLTELFLPASPLY

SEQ ID NO: 5 - protein, 473 aa, Bacillus subtilis
MAYQDFREFLAALEKEGQLLTVNEEVKPEPDLGASARAASNLGDKSPALLFNNIYGYHNARIAMNVIGSWPNHAMMLGMPKDTPVKEQFFEFAKRYDQFPMPVKREETAPFHENEITEDINLFDILPLFRINQGDGGYYLDKACVISRDLEDPDNFGKQNVGIYRMQVKGKDRLGIQPVPQHDIAIHLRQAEERGINLPVTIALGCEPVITTAASTPLLYDQSEYEMAGAIQGEPYRIVKSKLSDLDVPWGAEVVLEGEIIAGEREYEGPFGEFTGHYSGGRSMPIIKIKRVYHRNNPIFEHLYLGMPWTECDYMIGINTCVPLYQQLKEAYPNEIVAVNAMYTHGLIAIVSTKTRYGGFAKAVGMRALTTPHGLGYCKMVIVVDEDVDPFNLPQVMWALSTKMHPKHDAVIIPDLSVLPLDPGSNPSGITHKMILDATTPVAPETRGHYSQPLDSPLTTKEWEQKLMDLMNK

SEQ ID NO: 6 - protein, 350 aa, Escherichia coli
MNYQNDDLRIKEIKELLPPVALLEKFPATENAANTVAHARKAIHKILKGNDDRLLVVIGPCSIHDPVAAKEYATRLLALREELKDELEIVMRVYFEKPRTTVGWKGLINDPHMDNSFQINDGLRIARKLLLDINDSGLPAAGEFLDMITPQYLADLMSWGAIGARTTESQVHRELASGLSCPVGFKNGTDGTIKVAIDAINAAGAPHCFLSVTKWGHSAIVNTSGNGDCHIILRGGKEPNYSAKHVAEVKEGLNKAGLPAQVMIDFSHANSSKQFKKQMDVCADVCQQIAGGEKAIIGVMVESHLVEGNQSLESGEPLAYGKSITDACIGWEDTDALLRQLANAVKARRG

SEQ ID NO: 7 - protein, 395 aa, Pseudomonas putida
MKTQVAIIGAGPSGLLLGQLLHNAGIETVIVERQTPEYVLGRIRAGVLEQGTVDLLREAGVSARMDREGLVHEGVELLVGGRRQRLDLKALTGGKTVMVYGQTEVTRDLMQAREASGAPIIYAANNVQPHELKGERPYLTFEKDGQAHRLECDYIAGCDGFHGVSRQSIPEGVLKQYERVYPFGWLGLLSDTPPVNHELIYAHHERGFALCSQRSQTRSRYYLQVPLDDKVEAWSDERFWDELKARLPAEVAADLVTGPALEKSIAPLRSLVVEPMQYGHLFLVGDAAHIVPPTGAKGLNLAASDVNYLYRILVKVYGEGRTDLLQQYSPLALRRVWKGERFSWFMTQLLHDFGSHKDAWDQKMQEADREYFLNSPAGLLNIAENYVGLPYEAVV

SEQ ID NO: 8 - DNA, 32 bp, synthetic construct
  source 1..32: mol_type = other DNA
aggtctctcccgttttccagggtctgcaggcg

SEQ ID NO: 9 - DNA, 30 bp, synthetic construct
  source 1..30: mol_type = other DNA
aggtctctgcacagactcctatagtgaagc

SEQ ID NO: 10 - DNA, 35 bp, synthetic construct
  source 1..35: mol_type = other DNA
aggtctctgtgctccactcgtttcacagcacaagg

SEQ ID NO: 11 - DNA, 32 bp, synthetic construct
  source 1..32: mol_type = other DNA
aggtctcttcgacctggcagccatcatcgacc

SEQ ID NO: 12 - DNA, 31 bp, synthetic construct
  source 1..31: mol_type = other DNA
aggtctctcccgaggctgccgcggtcaaagc

SEQ ID NO: 13 - DNA, 30 bp, synthetic construct
  source 1..30: mol_type = other DNA
aggtctcttcatgattgggcagtctcaagg

SEQ ID NO: 14 - DNA, 28 bp, synthetic construct
  source 1..28: mol_type = other DNA
aggtctctatgagattgccgggggcgcg

SEQ ID NO: 15 - DNA, 33 bp, synthetic construct
  source 1..33: mol_type = other DNA
aggtctcttcgacaatatggccgaccagggcag

SEQ ID NO: 16 - DNA, 34 bp, synthetic construct
  source 1..34: mol_type = other DNA
aggtctctcccgtgctcaagttgtgcaggtgctg

SEQ ID NO: 17 - DNA, 26 bp, synthetic construct
  source 1..26: mol_type = other DNA
aggtctcttggcgctctctccgttgc

SEQ ID NO: 18 - DNA, 30 bp, synthetic construct
  source 1..30: mol_type = other DNA
aggtctctgccagggcaagcccgggtcatg

SEQ ID NO: 19 - DNA, 31 bp, synthetic construct
  source 1..31: mol_type = other DNA
aggtctcttcgacagctggccggcatccatg

SEQ ID NO: 20 - DNA, 30 bp, synthetic construct
  source 1..30: mol_type = other DNA
aggtctctcccgcaggttcgctacaagggc

SEQ ID NO: 21 - DNA, 30 bp, synthetic construct
  source 1..30: mol_type = other DNA
aggtctcttgttgttgtctctgttcggacg

SEQ ID NO: 22 - DNA, 27 bp, synthetic construct
  source 1..27: mol_type = other DNA
aggtctctaacagggctgcgagctacg

SEQ ID NO: 23 - DNA, 32 bp, synthetic construct
  source 1..32: mol_type = other DNA
aggtctcttcgatcctccttggggagcatttt

SEQ ID NO: 24 - DNA, 30 bp, synthetic construct
  source 1..30: mol_type = other DNA
aggtctctcccgcgaaggcgccgatcgggc

SEQ ID NO: 25 - DNA, 28 bp, synthetic construct
  source 1..28: mol_type = other DNA
aggtctctggcgggagttccttgttgtt

SEQ ID NO: 26 - DNA, 31 bp, synthetic construct
  source 1..31: mol_type = other DNA
aggtctctcgcctcgcctgcaccggcctgtc

SEQ ID NO: 27 - DNA, 32 bp, synthetic construct
  source 1..32: mol_type = other DNA
aggtctcttcgacacgctggtggatgcagcgc

SEQ ID NO: 28 - DNA, 32 bp, synthetic construct
  source 1..32: mol_type = other DNA
aggtctctcccgcctggccgttgatcggcccg

SEQ ID NO: 29 - DNA, 30 bp, synthetic construct
  source 1..30: mol_type = other DNA
aggtctctctgtcagtccttccgtttatgc

SEQ ID NO: 30 - DNA, 34 bp, synthetic construct
  source 1..34: mol_type = other DNA
aggtctctacagtcgcgccgcactgaagtcatgg

SEQ ID NO: 31 - DNA, 34 bp, synthetic construct
  source 1..34: mol_type = other DNA
aggtctcttcgacgtgggtcaggcaggtatcacc

SEQ ID NO: 32 - DNA, 32 bp, synthetic construct
  source 1..32: mol_type = other DNA
aggtctctcccgctgggggcagccagccgctg

SEQ ID NO: 33 - DNA, 30 bp, synthetic construct
  source 1..30: mol_type = other DNA
aggtctcttgggtgcatgggcttgatatcc

SEQ ID NO: 34 - DNA, 34 bp, synthetic construct
  source 1..34: mol_type = other DNA
aggtctctcccaatgaacgctctgaactacgaag

SEQ ID NO: 35 - DNA, 35 bp, synthetic construct
  source 1..35: mol_type = other DNA
aggtctcttcgactcgaccaggaagtcgaaacgtg

SEQ ID NO: 36 - DNA, 34 bp, synthetic construct
  source 1..34: mol_type = other DNA
aggtctctcccgttcgaagccggtcagtttgccg

SEQ ID NO: 37 - DNA, 30 bp, synthetic construct
  source 1..30: mol_type = other DNA
aggtctctggttttctcctgccgtataggc

SEQ ID NO: 38 - DNA, 35 bp, synthetic construct
  source 1..35: mol_type = other DNA
aggtctctaacctcccgcagtgcaattgcctgtgc

SEQ ID NO: 39 - DNA, 35 bp, synthetic construct
  source 1..35: mol_type = other DNA
aggtctcttcgacagacaaggcggtgaccaccacg

SEQ ID NO: 40 - DNA, 34 bp, synthetic construct
  source 1..34: mol_type = other DNA
aggtctctcccgatggtcgacgacatgaactggg

SEQ ID NO: 41 - DNA, 28 bp, synthetic construct
  source 1..28: mol_type = other DNA
aggtctctggctccacctaccttgtcgg

SEQ ID NO: 42 - DNA, 33 bp, synthetic construct
  source 1..33: mol_type = other DNA
aggtctctagccggcctatggcaacggtgatcg

SEQ ID NO: 43 - DNA, 36 bp, synthetic construct
  source 1..36: mol_type = other DNA
aggtctcttcgacgaagccacccacggcagcaaagg

SEQ ID NO: 44 - DNA, 36 bp, synthetic construct
  source 1..36: mol_type = other DNA
aggtctctcccgggctggatacctcgtcgctactgc

SEQ ID NO: 45 - DNA, 30 bp, synthetic construct
  source 1..30: mol_type = other DNA
aggtctctcgaccctgctcacgcttgacgg

SEQ ID NO: 46 - DNA, 35 bp, synthetic construct
  source 1..35: mol_type = other DNA
aggtctctgtcgttgccatctgaggacagcatgag

SEQ ID NO: 47 - DNA, 36 bp, synthetic construct
  source 1..36: mol_type = other DNA
aggtctcttcgacgcccggcgttcgtgcacaagttc

SEQ ID NO: 48 - DNA, 39 bp, synthetic construct
  source 1..39: mol_type = other DNA
aggtctctcatcctgctgaagagcatcatccacgactgg

SEQ ID NO: 49 - DNA, 31 bp, synthetic construct
  source 1..31: mol_type = other DNA
aggtctctgatggcgtcggccgacgggatcg

SEQ ID NO: 50 - DNA, 58 bp, synthetic construct
  source 1..58: mol_type = other DNA
aggtctctactagagaaagaggagaaatactagatgactaaatctgagcagcaagccg

SEQ ID NO: 51 - DNA, 43 bp, synthetic construct
  source 1..43: mol_type = other DNA
aggtctctgggcgtattattatgctaagtagctcagtaaatcc

SEQ ID NO: 52 - DNA, 54 bp, synthetic construct
  source 1..54: mol_type = other DNA
aggtctctgcccgaaagaggagaaatactagatgagccagcacgaccagcatcc

SEQ ID NO: 53 - DNA, 60 bp, synthetic construct
  source 1..60: mol_type = other DNA
taaggtctcgactgcagcggccgctactagtattattagatcttcgccagcgcctgcgcc

SEQ ID NO: 54 - DNA, 57 bp, synthetic construct
  source 1..57: mol_type = other DNA
tggtctcaccaagaaagaggagaaatactagatgaaagcagaattcaagcgtaaagg

SEQ ID NO: 55 - DNA, 48 bp, synthetic construct
  source 1..48: mol_type = other DNA
aggtctctggatgtattattaagctcctcctttttgtttttcaatccc

SEQ ID NO: 56 - DNA, 59 bp, synthetic construct
  source 1..59: mol_type = other DNA
tggtctcaatccgaaagaggagaaatactagatggcttatcaagatttcagagaatttc

SEQ ID NO: 57 - DNA, 48 bp, synthetic construct
  source 1..48: mol_type = other DNA
aggtctcttaccgtattattatttattcattaagtccattagtttttg

SEQ ID NO: 58 - DNA, 53 bp, synthetic construct
  source 1..53: mol_type = other DNA
tggtctcaggtagaaagaggagaaatactagatgcatacatgtcctcgatgcg

SEQ ID NO: 59 - DNA, 57 bp, synthetic construct
  source 1..57: mol_type = other DNA
taaggtctcgactgcagcggccgctactagtattattaagcctttcgttccggcacc

SEQ ID NO: 60 - DNA, 55 bp, synthetic construct
  source 1..55: mol_type = other DNA
aggtctctactagagaaagaggagaaatactagatgaattaccagaacgatgatc

SEQ ID NO: 61 - DNA, 39 bp, synthetic construct
  source 1..39: mol_type = other DNA
aggtctctgggcgtattattacccacgccgagctttcac

SEQ ID NO: 62 - DNA, 51 bp, synthetic construct
  source 1..51: mol_type = other DNA
aggtctctgcccgaaagaggagaaatactagatgtcacaccccgcgttaac

SEQ ID NO: 63 - DNA, 37 bp, synthetic construct
  source 1..37: mol_type = other DNA
aggtctctttgggtattattagtacaacggtgacgcc

SEQ ID NO: 64 - DNA, 33 bp, synthetic construct
  source 1..33: mol_type = other DNA
aggtctctccagtgatcaacacctgactacccg

SEQ ID NO: 65 - DNA, 36 bp, synthetic construct
  source 1..36: mol_type = other DNA
aggtctctacaaactggttttagagaattaaaactg

SEQ ID NO: 66 - DNA, 37 bp, synthetic construct
  source 1..37: mol_type = other DNA
aggtctcttagattattacgcgaacgcgaagtccgac

SEQ ID NO: 67 - DNA, 32 bp, synthetic construct
  source 1..32: mol_type = other DNA
aggtctctatgaacacgattaacatcgctaag

SEQ ID NO: 68 - DNA, 32 bp, synthetic construct
  source 1..32: mol_type = other DNA
aggtctctctggattctcaccaataaaaaacg

SEQ ID NO: 69 - DNA, 31 bp, synthetic construct
  source 1..31: mol_type = other DNA
aggtctctcttggactcctgttgatagatcc

SEQ ID NO: 70 - DNA, 33 bp, synthetic construct
  source 1..33: mol_type = other DNA
aggtctcttcatctagtatttctcctctttctc

SEQ ID NO: 71 - DNA, 52 bp, synthetic construct
  source 1..52: mol_type = other DNA
aggtctctggtactatagtgagtcgtattatggaattcgcggccgcttctag

SEQ ID NO: 72 - DNA, 35 bp, synthetic construct
  source 1..35: mol_type = other DNA
aggtctcttaccgttcgtatagcatacattatacg

SEQ ID NO: 73 - DNA, 32 bp, synthetic construct
  source 1..32: mol_type = other DNA
aggtctctttgtgcagcccgatgccgaagagg

SEQ ID NO: 74 - DNA, 34 bp, synthetic construct
  source 1..34: mol_type = other DNA
aggtctctcaagttataccagattgcgcagttcg

SEQ ID NO: 75 - DNA, 31 bp, synthetic construct
  source 1..31: mol_type = other DNA
aggtctcttctagagttgacggctagctcag